(19) United States
(12) Patent Application Publication
(10) Pub. No.: US 2024/0385183 A1
(43) Pub. Date: Nov. 21, 2024

(54) METABOLIC AND MICROBIAL BIOMARKERS FOR DETECTION OF PANCREATICOBILIARY CANCERS

(71) Applicant: Northwestern University, Evanston, IL (US)

(72) Inventors: Abdul Aziz AADAM, Evanston, IL (US); Jonathan XIA, Evanston, IL (US)

(21) Appl. No.: 18/669,411

(22) Filed: May 20, 2024

Related U.S. Application Data
(60) Provisional application No. 63/503,356, filed on May 19, 2023.

(51) Int. Cl. G01N 33/53; C12Q 1/06

(52) U.S. Cl. CPC G01N 33/5308; C12Q 1/06; G01N 2800/067; G01N 2800/7028

(57) ABSTRACT

Provided herein are biomarkers associated with pancreaticobiliary cancers. In particular, provided herein are metabolic, microbial, and/or glycomic biomarkers that, when measured in biological fluids (e.g., bile, serum, etc.) can be used to differentiate between pancreaticobiliary cancers and benign or other non-disease states.

### CROSS-REFERENCE TO RELATED APPLICATIONS

The present invention claims the priority benefit of U.S. Provisional Patent Application 63/503,356, filed May 19, 2023, which is incorporated by reference in its entirety.

### FIELD

Provided herein are biomarkers associated with pancreaticobiliary cancers. In particular, provided herein are metabolic, microbial, and/or glycomic biomarkers that, when measured in biological fluids (e.g., bile, serum, etc.) can be used to differentiate between pancreaticobiliary cancers and benign or other non-disease states.

### BACKGROUND

Cancers of the liver, bile ducts and pancreas are aggressive cancers with 5-year survival of 10-12%. One of the reasons that these cancers are so deadly is because there are no tests that can reliably detect these cancers in early stages. Furthermore, low diagnostic yield of currently available diagnostic methods (˜35%) also contributes to the late diagnosis of pancreaticobiliary cancers. Previous studies have shown that it can take up to 22 months from initial onset of symptoms to the diagnosis of these cancers, and more than one-third of patients are initially misdiagnosed with another type of cancer. Thus, accurate and early detection of pancreaticobiliary cancers is an unmet clinical need.

### SUMMARY

Provided herein are biomarkers associated with pancreaticobiliary cancers. In particular, provided herein are metabolic, microbial, and/or glycomic biomarkers that, when measured in biological fluids (e.g., bile, serum, etc.) can be used to differentiate between pancreaticobiliary cancers and benign or other non-disease states.

In some embodiments, provided herein are methods of assessing a pancreaticobiliary condition in a subject comprising assessing the level of one or more biomarkers in a bile sample from the subject.

In some embodiments, provided herein are methods of treating a subject suspected of suffering from an indeterminant biliary stricture, comprising: (a) assessing the levels of one or more biomarkers in a bile sample from the subject; (b) (i) if the biomarker level(s) indicate a malignancy, administering ERCP with cholangioscopy, endoscopic ultrasound guided biopsy, interventional radiology biopsy, PET CT to identify metastasis, or seeking further review of the subject, and (ii) if the biomarker level(s) indicate a benign condition, conducting additional imaging.

In some embodiments, provided herein are methods comprising assessing the level of three or more biomarkers in a bile sample from the subject, the biomarkers selected from: (a) one or more metabolomic biomarkers selected from the level of urea, norepinephrine, pyroglutamic acid, N-acetyl-DL-alanine, levulinic acid, uracil, hydroxyphenyllactic, 4-trimethylammoniobutanoate, ornithine, dihydroorotate, GalNAC/Glc/NAC/ManNAC, taurine, fructose mannose, AICA ribonucleotide, carbamoyl phosphate, 2-keto-isovaleric acid, N-acetyle-DL-serine, galactosamine/glucosamine, aconitic acid, mesaconic acid, itaconic acid, b-alanine, cystosine, lysine, citrate, methylguanine, phenethelamine, 3,4-Dihydroxyphenylacetic acid, D-galactonic acid, carnitine, quinolinic acid, orotic acid, lactic acid, dehydroascorbic acid, xanthine, hypoxanthine, guanine, glycine, 3-methoxytyramine, DL-DOPA, pantothenic acid, succinic acid, 3-aminoisobutanoate, y-glutamylcysteine, alpha-ketoglutarate, 3-phosphonatooxypyruvate, glyceric acid, alanine, creatine, 3-/7-methylguanine, sucrose, 5-methylcytosine, tryptamine, N-methyltryptamine, 1-phenylethylamine, pyruvic acid, deoxyadenosine, deoxycytidine triphosphate (dCTP), picolinic acid, guanidinoacetate, NADPH, 5′methylthioadenosine, deoxyuridine, D-sedoheptulose-7-phoisphate, aspartic acid, lactose/maltose, uric acid, phenylalanine, glutamate, uridine monophosphate (UMP), uracil, and xanthine; (b) one or more microbiomic biomarkers selected from the level of bacteria belonging to one or more genera selected from Peptostreptococcus, Neisseria, Absconditabacteria (SR1), Leptotrichia, Gemella, Lachnospiraceae, Lachnoanaerobaculum, Moigibacterium, Megasphaera, Comamonadaceae, Enhydrobacter, Escherichia-Shigella, Enterococcus, Streptococcus, Actinomyces, and Veillonella; and/or (c) one or more glycomic biomarkers and selected from HexNAc₄Hex₄Fuc₃, HexNAc4Hex5NeuAc1, HexNAc4Hex4Fuc4, HexNAc5Hex4Fuc3, HexNAc4Hex5NeuAc2, HexNAc5Hex4Fuc4, HexNAc3Hex3Fuc2, HexNAc4Hex2Fuc2, HexNAc4Hex3Fuc1, HexNAc4Hex4, HexNAc5Hex3, HexNAc3Hex2NeuAc1Fuc2, HexNAc3Hex3Fuc3, HexNAc3Hex3NeuAc1Fuc1, HexNAc2Hex4Fuc3, HexNAc4Hex3Fuc2, HexNAc4Hex4Fuc1, HexNAc5Hex3Fuc1, HexNAc3Hex3Fuc4, HexNAc3Hex3NeuAc1Fuc2, HexNAc4Hex3Fuc3, HexNAc4Hex4Fuc2, HexNAc5Hex3Fuc2, HexNAc5Hex4Fuc1, HexNAc4Hex3Fuc4, HexNAc4Hex3NeuAc1Fuc2, HexNAc2Hex2NeuAc1, HexNAc2Hex3Fuc1, HexNAc3Hex2Fuc1, HexNAc3Hex3Fuc1, HexNAc4Hex2, HexNAc2Hex2Fuc3, HexNAc2Hex2NeuAc1Fuc1, HexNAc2Hex3Fuc2, HexNAc3Hex2Fuc2, HexNAc3Hex2NeuAc1, HexNAc3Hex3Fuc1, HexNAc1Hex3NeuAc1Fuc2, HexNAc1Hex3NeuAc1, HexNAc4Hex3, HexNAc2Hex2NeuAc1Fuc2, HexNAc2Hex2NeuAc2, HexNAc2Hex3NeuAc1Fuc1, HexNAc2Hex4Fuc2, HexNAc3Hex2Fuc3, HexNAc3Hex2NeuAc1Fuc1, HexNAc1Hex1, HexNAc2, HexNAc1NeuAc1, HexNAc1Hex1Fuc1, HexNAc2Hex1, HexNAc1Hex1NeuAc1, HexNAc2NeuAc1, HexNAc2Hex1Fuc1, HexNAc2Hex2, HexNAc3Hex1, HexNAc1Hex1NeuAc1Fuc1, HexNAc1Hex2Fuc2, HexNAc2Hex1NeuAc1, HexNAc2Hex2Fuc1, HexNAc3Hex1Fuc1, HexNAc3Hex2, HexNAc1Hex1NeuAc2, HexNAc1Hex3NeuAc1, HexNAc2Hex1NeuAc1Fuc1, HexNAc2Hex2Fuc2, (Hex)3 (HexNAc)3 (Deoxyhexose)1 (NeuAc)3+(Man)3(GlcNAc)2, (Hex)3 (HexNAc)3 (Deoxyhexose)2 (NeuAc)3+(Man)3(GlcNAc)2, (Hex)4 (HexNAc)4 (NeuAc)3+(Man)3(GlcNAc)2, (Hex)4 (HexNAc)4 (Deoxyhexose)1 (NeuAc)3+(Man)3(GlcNAc)2, (Hex)4 (HexNAc)4 (NeuAc)4+(Man)3(GlcNAc)2, (Hex)4 (HexNAc)4 (Deoxyhexose)1 (NeuAc)4+(Man)3(GlcNAc)2, (Hex)2 (HexNAc)2 (NeuAc)1 (NeuGc)1+(Man)3(GlcNAc)2, (Hex)2 (HexNAc)3 (Deoxyhexose)1 (NeuAc)1+, (Man)3(GlcNAc)2, (Hex)3 (HexNAc)3 (NeuAc)1+(Man)3(GlcNAc)2, (Hex)2 (HexNAc)4 (Deoxyhexose)2+(Man)3(GlcNAc)2, (Hex)2 (HexNAc)2 (Deoxyhexose)3 (NeuAc)1+(Man)3(GlcNAc)2, (Hex)2 (HexNAc)2 (Deoxyhexose)1 (NeuAc)2+(Man)3(GlcNAc)2, (Hex)3 (HexNAc)2 (NeuAc)2+(Man)3(GlcNAc)2 (Hex)2 (HexNAc)3 (Deoxyhexose)4+(Man)3(GlcNAc)2, (Hex)2 (HexNAc)3 (Deoxyhexose)2 (NeuAc)1+(Man)3(GlcNAc)2, (Hex)3 (HexNAc)3 (Deoxyhexose)1 (NeuAc)1+(Man)3(GlcNAc)2, (Hex)2 (HexNAc)4 (Deoxyhexose)3+(Man)3(GlcNAc)2, (Hex)2 (HexNAc)3 (Deoxyhexose)3 (NeuAc)1+(Man)3(GlcNAc)2, (Hex)2 (HexNAc)3 (Deoxyhexose)1 (NeuAc)2+(Man)3(GlcNAc)2, (Hex)3 (HexNAc)3 (NeuAc)2+(Man)3(GlcNAc)2, (Hex)3 (HexNAc)4 (Deoxyhexose)3+(Man)3(GlcNAc)2, (Hex)3 (HexNAc)3 (Deoxyhexose)1 (NeuAc)2+(Man)3(GlcNAc)2, (Hex)3 (HexNAc)4 (Deoxyhexose)4+(Man)3(GlcNAc)2, (Hex)3 (HexNAc)3 (NeuAc)3+(Man)3(GlcNAc)2, (Hex)3 (HexNAc)4 (Deoxyhexose)3 (NeuAc)1+(Man)3(GlcNAc)2 (Hex)4 (HexNAc)4 (NeuAc)2+(Man)3(GlcNAc)2, (Hex)6+(Man)3(GlcNAc)2, (Hex)1 (HexNAc)2 (Deoxyhexose)1 (NeuAc)1+(Man)3(GlcNAc)2, (Hex)2 (HexNAc)2 (NeuAc)1+(Man)3(GlcNAc)2, (Hex)1 (HexNAc)3 (Deoxyhexose)2+(Man)3(GlcNAc)2, (Hex)1 (HexNAc)3 (NeuAc)1+(Man)3(GlcNAc)2, (Hex)2 (HexNAc)3 (Deoxyhexose)1+(Man)3(GlcNAc)2, (Hex)2 (HexNAc)1 (NeuAc)2+(Man)3(GlcNAc)2, (Hex)2 (HexNAc)2 (Deoxyhexose)1 (NeuAc)1+(Man)3(GlcNAc)2, (Hex)1 (HexNAc)3 (Deoxyhexose)3+(Man)3(GlcNAc)2, (Hex)1 (HexNAc)3 (Deoxyhexose)1 (NeuAc)1+(Man)3(GlcNAc)2, (Hex)2 (HexNAc)3 (Deoxyhexose)2+(Man)3(GlcNAc)2, (Hex)2 (HexNAc)3 (NeuAc)1+(Man)3(GlcNAc)2, (Hex)5 (HexNAc)2+(Man)3(GlcNAc)2, (Hex)2 (HexNAc)1 (Deoxyhexose)1 (NeuAc)2+(Man)3(GlcNAc)2, (Hex)1 (HexNAc)5 (Deoxyhexose)5+(Man)3(GlcNAc)2, (Hex)1 (HexNAc)2 (Deoxyhexose)1 (NeuAc)2+(Man)3(GlcNAc)2, (Hex)1 (HexNAc)5 (Deoxyhexose)1+(Man)3(GlcNAc)2, (Hex)2 (HexNAc)2 (NeuAc)2+(Man)3(GlcNAc)2, (Hex)2 (HexNAc)5+(Man)3(GlcNAc)2, (Hex)3 (HexNAc)2 (Deoxyhexose)1 (NeuAc)1+(Man)3(GlcNAc)2, (Hex)2 (HexNAc)2, (Man)3(GlcNAc)2, (Hex)2+(Man)3(GlcNAc)2, (HexNAc)2+(Man)3(GlcNAc)2, (Hex)3+(Man)3(GlcNAc)2, (HexNAc)2 (Deoxyhexose)1+(Man)3(GlcNAc)2, (HexNAc)3+(Man)3(GlcNAc)2, (Hex)1 (HexNAc)1 (NeuAc)1+(Man)3(GlcNAc)2, (Hex)4+(Man)3(GlcNAc)2, (Hex)1 (HexNAc)2 (Deoxyhexose)1+(Man)3(GlcNAc)2, (Hex)2 (HexNAc)2+(Man)3(GlcNAc)2, (HexNAc)3 (Deoxyhexose)1+(Man)3(GlcNAc)2, (Hex)1 (HexNAc)3+(Man)3(GlcNAc)2, (Hex)2 (HexNAc)1 (NeuAc)1+(Man)3(GlcNAc)2, (Hex)5+(Man)3(GlcNAc)2, (Hex)1 (HexNAc)2 (Deoxyhexose)2+(Man)3(GlcNAc)2, (Hex)1 (HexNAc)2 (NeuAc)1+(Man)3(GlcNAc)2, (Hex)2 (HexNAc)2 (Deoxyhexose)1+(Man)3(GlcNAc)2, (Hex)1 (HexNAc)3 (Deoxyhexose)1+(Man)3(GlcNAc)2, and (Hex)3 (HexNAc)1 (NeuAc)1+(Man)3(GlcNAc)2.

In some embodiments, provided herein are methods comprising assessing the level of three or more of 3-methylguanine, 5-methylcytosine, butyric acid, and urea in a bile sample from the subject.

In some embodiments, the subject suffers exhibits one or more symptoms of the pancreaticobiliary condition. In some embodiments, the symptoms of the pancreaticobiliary condition are selected from abdominal pain, loss of appetite, weight loss, jaundice, discolored urine or stool, itchy skin, diabetes, and blood clots. In some embodiments, the subject has received test results indicating a possible pancreaticobiliary condition. In some embodiments, the test results are selected from imaging and a blood test. In some embodiments, assessing a pancreaticobiliary condition in a subject comprises determining the likelihood that a subject suffers from a pancreaticobiliary cancer versus a benign or non-cancerous pancreaticobiliary condition. In some embodiments, a pancreaticobiliary cancer is selected from a cancer of the pancreas gland, pancreas duct, ampulla, bile ducts, gallbladder, liver parenchyma, duodenum, dominant stricture in patient with primary sclerosing cholangitis, and enlarged lymph nodes in the peri-portal region. In some embodiments, a benign or non-cancerous pancreaticobiliary condition is selected from a benign stricture, acute or chronic pancreatitis (or both), pancreatic obstruction, choledocholithiasis, Mirizzi syndrome, primary sclerosing cholangitis, liver transplant related anastomotic stricture, biliary surgery related stricture, biliary anatomic variations.autoimmune pancreatitis and cholangitis etc. In some embodiments, the one or more biomarkers are selected from microbiomic, metabolomic, and/or glycomic biomarkers.

In some embodiments, the one or more microbiomic biomarkers are selected from the level of bacteria belonging to one or more genera selected from Peptostreptococcus, Neisseria, Absconditabacteria (SR1), Leptotrichia, Gemella, Lachnospiraceae, Lachnoanaerobaculum, Moigibacterium, Megasphaera, Comamonadaceae, Enhydrobacter, Escherichia-Shigella, Enterococcus, Streptococcus, Actinomyces, and Veillonella. In some embodiments, an increased level of one or more of Peptostreptococcus, Neisseria, Absconditabacteria (SR1), Leptotrichia, Gemella, Lachnospiraceae, Lachnoanaerobaculum, and/or Moigibacterium is associated with increased risk of one or more pancreaticobiliary cancers. In some embodiments, a decreased level of one or more of Megasphaera, Comamonadaceae, Enhydrobacter, Escherichia-Shigella, Enterococcus, Streptococcus, Actinomyces, Veillonella is associated with increased risk of one or more pancreaticobiliary cancers. In some embodiments, the level of one or more microbiome biomarkers is assessed by nucleic acid sequencing.

In some embodiments, the level of one or more metabolomic biomarkers is assessed by one or more biophysical techniques. In some embodiments, the level of one or more metabolomic biomarkers is assessed by mass spectrometric analysis of the bile sample from the subject. In some embodiments, the one or more metabolomic biomarkers and selected from the level of urea, norepinephrine, pyroglutamic acid, N-acetyl-DL-alanine, levulinic acid, uracil, hydroxyphenyllactic, 4-trimethylammoniobutanoate, ornithine, dihydroorotate, GalNAC/Glc/NAC/ManNAC, taurine, fructose mannose, AICA ribonucleotide, carbamoyl phosphate, 2-keto-isovaleric acid, N-acetyle-DL-serine, galactosamine/glucosamine, aconitic acid, mesaconic acid, itaconic acid, b-alanine, cystosine, lysine, citrate, methylguanine, phenethelamine, 3,4-Dihydroxyphenylacetic acid, D-galactonic acid, carnitine, quinolinic acid, orotic acid, lactic acid, dehydroascorbic acid, xanthine, hypoxanthine, guanine, glycine, 3-methoxytyramine, DL-DOPA, pantothenic acid, succinic acid, 3-aminoisobutanoate, y-glutamylcysteine, alpha-ketoglutarate, 3-phosphonatooxypyruvate, glyceric acid, alanine, and/or creatine. In some embodiments, an increased level of one or more of urea, norepinephrine, pyroglutamic acid, N-acetyl-DL-alanine, levulinic acid, uracil, hydroxyphenyllactic, 4-trimethylammoniobutanoate, ornithine, dihydroorotate, GalNAC/Glc/NAC/ManNAC, taurine, fructose mannose, AICA ribonucleotide, carbamoyl phosphate, 2-keto-isovaleric acid, N-acetyle-DL-serine, galactosamine/glucosamine, aconitic acid, mesaconic acid, itaconic acid, b-alanine, cystosine, lysine, citrate, methylguanine, phenethelamine, 3,4-Dihydroxyphenylacetic acid, D-galactonic acid, carnitine, quinolinic acid, orotic acid, lactic acid, dehydroascorbic acid, xanthine, hypoxanthine, and/or guanine is associated with increased risk of one or more pancreaticobiliary cancers. In some embodiments, a decreased level of one or more of glycine, 3-methoxytyramine, DL-DOPA, pantothenic acid, succinic acid, 3-aminoisobutanoate, y-glutamylcysteine, alpha-ketoglutarate, 3-phosphonatooxypyruvate, glyceric acid, alanine, and creatine is associated with increased risk of one or more pancreaticobiliary cancers.

In some embodiments, the level of one or more glycomic biomarkers is assessed by one or more biophysical techniques. In some embodiments, the level of one or more glycomic biomarkers is assessed by mass spectrometric analysis of the bile sample from the subject. In some embodiments, the one or more glycomic biomarkers and selected from HexNAc₄Hex₄Fuc₃, HexNAc4Hex5NeuAc1, HexNAc4Hex4Fuc4, HexNAc5Hex4Fuc3, HexNAc4Hex5NeuAc2, HexNAc5Hex4Fuc4, HexNAc3Hex3Fuc2, HexNAc4Hex2Fuc2, HexNAc4Hex3Fuc1, HexNAc4Hex4, HexNAc5Hex3, HexNAc3Hex2NeuAc1Fuc2, HexNAc3Hex3Fuc3, HexNAc3Hex3NeuAc1Fuc1, HexNAc2Hex4Fuc3, HexNAc4Hex3Fuc2, HexNAc4Hex4Fuc1, HexNAc5Hex3Fuc1, HexNAc3Hex3Fuc4, HexNAc3Hex3NeuAc1Fuc2, HexNAc4Hex3Fuc3, HexNAc4Hex4Fuc2, HexNAc5Hex3Fuc2, HexNAc5Hex4Fuc1, HexNAc4Hex3Fuc4, HexNAc4Hex3NeuAc1Fuc2, HexNAc2Hex2NeuAc1, HexNAc2Hex3Fuc1, HexNAc3Hex2Fuc1, HexNAc3Hex3Fuc1, HexNAc4Hex2, HexNAc2Hex2Fuc3, HexNAc2Hex2NeuAc1Fuc1, HexNAc2Hex3Fuc2, HexNAc3Hex2Fuc2, HexNAc3Hex2NeuAc1, HexNAc3Hex3Fuc1, HexNAc1Hex3NeuAc1Fuc2, HexNAc1Hex3NeuAc1, HexNAc4Hex3, HexNAc2Hex2NeuAc1Fuc2, HexNAc2Hex2NeuAc2, HexNAc2Hex3NeuAc1Fuc1, HexNAc2Hex4Fuc2, HexNAc3Hex2Fuc3, HexNAc3Hex2NeuAc1Fuc1, HexNAc1Hex1, HexNAc2, HexNAc1NeuAc1, HexNAc1Hex1Fuc1, HexNAc2Hex1, HexNAc1Hex1NeuAc1, HexNAc2NeuAc1, HexNAc2Hex1Fuc1, HexNAc2Hex2, HexNAc3Hex1, HexNAc1Hex1NeuAc1Fuc1, HexNAc1Hex2Fuc2, HexNAc2Hex1NeuAc1, HexNAc2Hex2Fuc1, HexNAc3Hex1Fuc1, HexNAc3Hex2, HexNAc1Hex1NeuAc2, HexNAc1Hex3NeuAc1, HexNAc2Hex1NeuAc1Fuc1, HexNAc2Hex2Fuc2, (Hex)3 (HexNAc)3 (Deoxyhexose)1 (NeuAc)3+(Man)3(GlcNAc)2, (Hex)3 (HexNAc)3 (Deoxyhexose)2 (NeuAc)3+(Man)3(GlcNAc)2, (Hex)4 (HexNAc)4 (NeuAc)3+(Man)3(GlcNAc)2, (Hex)4 (HexNAc)4 (Deoxyhexose)1 (NeuAc)3+(Man)3(GlcNAc)2, (Hex)4 (HexNAc)4 (NeuAc)4+(Man)3(GlcNAc)2, (Hex)4 (HexNAc)4 (Deoxyhexose)1 (NeuAc)4+(Man)3(GlcNAc)2, (Hex)2 (HexNAc)2 (NeuAc)1 (NeuGc)1+(Man)3(GlcNAc)2, (Hex)2 (HexNAc)3 (Deoxyhexose)1 (NeuAc)1+, (Man)3(GlcNAc)2, (Hex)3 (HexNAc)3 (NeuAc)1+(Man)3(GlcNAc)2, (Hex)2 (HexNAc)4 (Deoxyhexose)2+(Man)3(GlcNAc)2, (Hex)2 (HexNAc)2 (Deoxyhexose)3 (NeuAc)1+(Man)3(GlcNAc)2, (Hex)2 (HexNAc)2 (Deoxyhexose)1 (NeuAc)2+(Man)3(GlcNAc)2, (Hex)3 (HexNAc)2 (NeuAc)2+(Man)3(GlcNAc)2, (Hex)2 (HexNAc)3 (Deoxyhexose)4+(Man)3(GlcNAc)2, (Hex)2 (HexNAc)3 (Deoxyhexose)2 (NeuAc)1+(Man)3(GlcNAc)2, (Hex)3 (HexNAc)3 (Deoxyhexose)1 (NeuAc)1+(Man)3(GlcNAc)2, (Hex)2 (HexNAc)4 (Deoxyhexose)3+(Man)3(GlcNAc)2, (Hex)2 (HexNAc)3 (Deoxyhexose)3 (NeuAc)1+(Man)3(GlcNAc)2, (Hex)2 (HexNAc)3 (Deoxyhexose)1 (NeuAc)2+(Man)3(GlcNAc)2, (Hex)3 (HexNAc)3 (NeuAc)2+(Man)3(GlcNAc)2, (Hex)3 (HexNAc)4 (Deoxyhexose)3+(Man)3(GlcNAc)2, (Hex)3 (HexNAc)3 (Deoxyhexose)1 (NeuAc)2+(Man)3(GlcNAc)2, (Hex)3 (HexNAc)4 (Deoxyhexose)4+(Man)3(GlcNAc)2, (Hex)3 (HexNAc)3 (NeuAc)3+(Man)3(GlcNAc)2, (Hex)3 (HexNAc)4 (Deoxyhexose)3 (NeuAc)1+(Man)3(GlcNAc)2, (Hex)4 (HexNAc)4 (NeuAc)2+(Man)3(GlcNAc)2, (Hex)6+(Man)3(GlcNAc)2, (Hex)1 (HexNAc)2 (Deoxyhexose)1 (NeuAc)1+(Man)3(GlcNAc)2, (Hex)2 (HexNAc)2 (NeuAc)1+(Man)3(GlcNAc)2, (Hex)1 (HexNAc)3 (Deoxyhexose)2+(Man)3(GlcNAc)2, (Hex)1 (HexNAc)3 (NeuAc)1+(Man)3(GlcNAc)2, (Hex)2 (HexNAc)3 (Deoxyhexose)1+(Man)3(GlcNAc)2, (Hex)2 (HexNAc)1 (NeuAc)2+(Man)3(GlcNAc)2, (Hex)2 (HexNAc)2 (Deoxyhexose)1 (NeuAc)1+(Man)3(GlcNAc)2, (Hex)1 (HexNAc)3 (Deoxyhexose)3+(Man)3(GlcNAc)2, (Hex)1 (HexNAc)3 (Deoxyhexose)1 (NeuAc)1+(Man)3(GlcNAc)2, (Hex)2 (HexNAc)3 (Deoxyhexose)2+(Man)3(GlcNAc)2, (Hex)2 (HexNAc)3 (NeuAc)1+(Man)3(GlcNAc)2, (Hex)5 (HexNAc)2+(Man)3(GlcNAc)2, (Hex)2 (HexNAc)1 (Deoxyhexose)1 (NeuAc)2+(Man)3(GlcNAc)2, (Hex)1 (HexNAc)5 (Deoxyhexose)5+(Man)3(GlcNAc)2, (Hex)1 (HexNAc)2 (Deoxyhexose)1 (NeuAc)2+(Man)3(GlcNAc)2, (Hex)1 (HexNAc)5 (Deoxyhexose)1+(Man)3(GlcNAc)2, (Hex)2 (HexNAc)2 (NeuAc)2+(Man)3(GlcNAc)2, (Hex)2 (HexNAc)5+(Man)3(GlcNAc)2, (Hex)3 (HexNAc)2 (Deoxyhexose)1 (NeuAc)1+(Man)3(GlcNAc)2, (Hex)2 (HexNAc)2, (Man)3(GlcNAc)2, (Hex)2+(Man)3(GlcNAc)2, (HexNAc)2+(Man)3(GlcNAc)2, (Hex)3+(Man)3(GlcNAc)2, (HexNAc)2 (Deoxyhexose)1+(Man)3(GlcNAc)2, (HexNAc)3+(Man)3(GlcNAc)2, (Hex)1 (HexNAc)1 (NeuAc)1+(Man)3(GlcNAc)2, (Hex)4+(Man)3(GlcNAc)2, (Hex)1 (HexNAc)2 (Deoxyhexose)1+(Man)3(GlcNAc)2, (Hex)2 (HexNAc)2+(Man)3(GlcNAc)2, (HexNAc)3 (Deoxyhexose)1+(Man)3(GlcNAc)2, (Hex)1 (HexNAc)3+(Man)3(GlcNAc)2, (Hex)2 (HexNAc)1 (NeuAc)1+(Man)3(GlcNAc)2, (Hex)5+(Man)3(GlcNAc)2, (Hex)1 (HexNAc)2 (Deoxyhexose)2+(Man)3(GlcNAc)2, (Hex)1 (HexNAc)2 (NeuAc)1+(Man)3(GlcNAc)2, (Hex)2 (HexNAc)2 (Deoxyhexose)1+(Man)3(GlcNAc)2, (Hex)1 (HexNAc)3 (Deoxyhexose)1+(Man)3(GlcNAc)2, and (Hex)3 (HexNAc)1 (NeuAc)1+(Man)3(GlcNAc)2. In some embodiments, an increased level of one or more of HexNAc4Hex4Fuc4, HexNAc5Hex3Fuc2, and/or HexNAc3Hex2Fuc3 is associated with increased risk of one or more pancreaticobiliary cancers.

In some embodiments, the level of one or more biomarkers in the bile sample are compared to a threshold level. In some embodiments, the level of one or more biomarkers in the bile sample are compared to previous levels for the subject.

In some embodiments, methods further comprise obtaining a bile sample from the subject. In some embodiments, the bile sample is obtained during endoscopic retrograde cholangiopancreatography (ERCP). In some embodiments, the bile sample is a fluid biopsy sample. In some embodiments, the bile sample is obtained during a procedure to obtain a solid biopsy sample of tissue and/or cells from the subject. In some embodiments, the bile sample is obtained without a solid biopsy sample. In some embodiments, the bile sample is aspirated using a sphincterotome after deep bile cannulation. In some embodiments, the bile sample is 0.1 to 5.0 ml in volume.

In some embodiments, assessing the pancreaticobiliary condition in the subject further comprises imaging, endoscopic ultrasound, fluorescence in-situ hybridization, cytology, and/or histology.

In some embodiments, methods further comprise treating a subject for the pancreaticobiliary condition. In some embodiments, assessing the pancreaticobiliary condition indicates that the subject suffers from a pancreaticobiliary malignancy and the subject is treated for the pancreaticobiliary cancer. In some embodiments, treating the subject for the pancreaticobiliary cancer comprises surgery, radiation treatment, and/or administering a chemotherapeutic or immunotherapeutic. In some embodiments, assessing the pancreaticobiliary condition indicates that the subject does not suffer from a pancreaticobiliary malignancy and the subject is treated for a benign and/or non-cancerous pancreaticobiliary condition.

### BRIEF DESCRIPTION OF THE DRAWINGS

FIG. 1. Bile metabolites are differentially abundant in patients with pancreaticobiliary tumors compared to benign biliary disease. Heat map showing the top statistically significant differentially abundant metabolites between bile samples in patients with benign biliary disease and new pancreaticobiliary cancers.

FIG. 2. Unbiased hierarchical clustering a test subjects based on metabolic screening.

FIG. 3. Distinct bile microbiome composition for patients with different biliary conditions.

FIG. 4. hierarchical clustering of phylogenetic distances of different biliary conditions.

FIG. 5. Bile microbiome composition is distinct in patients with malignant biliary strictures compared patients with benign biliary disease and PSC.

FIG. 6. Analysis of the microbiome (ANCOM) showed differentially abundant organisms associated with malignant strictures. ANCOM is a statistic that can identify specific bacteria groups that are either significantly increased or decreased in comparisons of different groups. ANCOM was used to compare the microbial communities in bile from patients with malignancies and those with PSC and benign biliary disease.

FIG. 7. ANCOM showed differentially abundant organisms associated with malignant strictures.

FIG. 8A-D. MALDI-TOF spectra of N-glycans from control (A/B) and cancer (C/D) patients.

FIG. 9A-D. N-glycan compositional comparison table.

FIG. 10. N-glycan compositional comparison graphs.

FIG. 11A-D. MALDI-TOF spectra of O-glycans from control (A/B) and cancer (C/D) patients.

FIG. 12A-D. O-glycan compositional comparison table.

FIG. 13. O-glycan compositional comparison graphs.

FIG. 14. The Biliary metabolome is distinct in patients with cholangiocarcinoma.

FIG. 15. Bile Metabolomics identified clear differences in the abundance of metabolites in patients with cholangiocarcinoma.

FIG. 16. Bile metabolomics identified distinct metabolite signatures of cholangiocarcinoma.

FIG. 17. Bile metabolomics identified distinct metabolite signatures of cholangiocarcinoma.

FIG. 18. Prediction of cholangiocarcinoma through bile levels of an exemplary 4-metabolite panel (5-methylcytosine, 3/7-methyl guanine, butyrate, urea).

FIG. 19. Example of diagnostic workflow of bile fluid metabolite analysis to enhance endoscopic work up of indeterminate biliary strictures.

FIG. 20. Absolute quantification curve for exemplary metabolite panel.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a metabolite” is a reference to one or more metabolites and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “microbiome” refers to the microbes (e.g., bacteria, fungi, protists) their genetic elements (genomes) in a defined environment. A “microbimic analysis” refers to the use of any suitable techniques to identify the presence/absence/level of one or more (e.g., all) microbes within a sample or environment. The term “microbiomic biomarker” refers to a species or group of microbes, the presence/absence/level of which in a sample from a subject is indicative/prognostic/diagnostic of a condition/outcome in the subject.

As used herein, the term “metabolome” refers to the complete set of small-molecule metabolites (e.g., metabolic intermediates, hormones signaling molecules, secondary metabolites, etc.) found within a defined biological environment or sample. A “metabolomic analysis” refers to the use of any suitable techniques to identify the presence/absence/level of one or more (e.g., all) of the metabolites within a sample or environment. The term “metabolomic biomarker” refers to a small molecule metabolite, the presence/absence/level of which in a sample from a subject is indicative/prognostic/diagnostic of a condition/outcome in the subject.

As used herein, the term “glycome” refers to the complete repertoire of glycans and glycoconjugates that cells produce under specified conditions of time, space, and environment. A “glycomic analysis” refers to the use of any suitable techniques to identify the presence/absence/level of one or more (e.g., all) glycosylation and glycan markers within a sample or environment. The term “glycomic biomarker” refers to a glycosylation or glycan marker, the presence/absence/level of which in a sample from a subject is indicative/prognostic/diagnostic of a condition/outcome in the subject.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, the term “sample” or “biological sample” refers to any material, fluid, cells, tissues, etc. removed, obtained, or extracted from a subject. A sample further may include a homogenate, lysate, extract, fraction, etc. prepared from a whole organism or a subset of its tissues, cells, fluids, or component parts. Examples of biological samples include blood (including whole blood, leukocytes, peripheral blood mononuclear cells, buffy coat, plasma, and serum), sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, bronchial brushing, synovial fluid, joint aspirate, organ secretions, cells, a cellular extract, and cerebrospinal fluid. In particular, a biological sample comprises bile. This also includes experimentally separated fractions, homogenizations, or other processed variants of all of the preceding.

As used herein, the term “bile” refers to a liquid substance produced by the liver comprising water, bile salts, mucus, fats, inorganic salts, cholesterol, and other metabolites, which aids in the emulsification and digestion of dietary fats.

As used herein, “marker” and “biomarker” are used interchangeably to refer to a target molecule (e.g., metabolite) or entity (e.g., complex or microbe) that indicates or is a sign of a normal or abnormal process in an individual or of a disease or other condition in an individual. Biomarkers are detectable and measurable by a variety of methods including laboratory assays.

As used herein, “biomarker value”, “value”, “biomarker level”, and “level” are used interchangeably to refer to a measurement that is made using any analytical method for detecting the biomarker in a biological sample and that indicates the presence, absence, absolute amount or concentration, relative amount or concentration, titer, a level, an expression level, a ratio of measured levels, or the like, of, for, or corresponding to the biomarker in the biological sample. The exact nature of the “value” or “level” depends on the specific design and components of the particular analytical method employed to detect the biomarker.

When a biomarker indicates or is a sign of an abnormal process or a disease or other condition in an individual, that biomarker may generally described herein as being either “upregulated or “downregulated” level or value of the biomarker. “Upregulation,” and any variations thereof refer to a value or level of a biomarker in a biological sample that is greater than a value or level (or range of values or levels) of the biomarker that is detected in a control or compared to a threshold (e.g., similar biological samples from healthy or normal individuals). The terms may also refer to a value or level of a biomarker in a biological sample that is greater than a value or level (or range of values or levels) of the biomarker (e.g., a threshold). Conversely, “downregulation”, and any variations thereof refers to a value or level of a biomarker in a biological sample that is less than a value or level (or range of values or levels) of the biomarker in a control (e.g., in similar biological samples from healthy or normal individuals). The terms may also refer to a value or level of a biomarker in a biological sample that is less than a value or level (or range of values or levels) of the biomarker (e.g., a threshold).

As used herein, the term “diagnosis” refers to the detection, determination, or recognition of a health status or condition of an individual on the basis of one or more signs, symptoms, data, or other information pertaining to that individual. The terms “diagnose”, “diagnosing”, “diagnosis”, etc., encompass, with respect to a particular disease or condition, the initial detection of the disease; the characterization or classification of the disease; the detection of the progression, remission, or recurrence of the disease; and the detection of disease response after the administration of a treatment or therapy to the individual. The diagnosis of pancreatic cancer includes distinguishing individuals who have cancer from individuals who do not. It further includes distinguishing GI and normal controls from pancreatic cancer.

As used herein, the term “prognosis” refers to risk prediction of the severity of disease or of the probable course and clinical outcome associated with a disease. Thus, the term “method of prognosis” as used herein refers to methods by which the skilled person can estimate and/or determine a probability that a given outcome will occur.

The terms “stratification” or “stratifying” as used herein refer to the division of a population into subpopulations on the basis of specified criteria. More particularly, it refers to the division of a cohort of subjects into at least two groups on the basis of specific criteria, which in the context of the present invention comprise or consist of the results of the method of analysis.

As used herein, the term “pancreaticobiliary” refers to tissues and organs including, but not limited to, the pancreas gland, pancreas duct, pancreatic ampulla, bile ducts, gallbladder, liver parenchyma, and duodenum.

### DETAILED DESCRIPTION

Provided herein are biomarkers associated with pancreaticobiliary cancers. In particular, provided herein are metabolic, microbial, and/or glycomic biomarkers that, when measured in biological fluids (e.g., bile, serum, etc.) can be used to differentiate between pancreaticobiliary cancers and benign or other non-disease states.

According to the Surveillance, Epidemiology, and End Results (SEER) database, the prevalence of cholangiocarcinoma in 2020 was 105,765, with an estimated 41,210 new cases per year. This accounts for 2.1% of all new cancer cases in the United States. One of the earliest signs of bile duct cancer is presence of bile duct narrowing, biliary stricture. Current modalities on differentiating benign and malignant strictures have modest diagnostic sensitivity of 35-48%.

Thus, identification of reliable and sensitive clinical diagnostic biomarker of bile duct malignancies is currently an unmet clinical need. In some embodiments, provided herein are methods for differentiating between benign and malignant strictures have limited diagnostic sensitivity, which often results in multiple rounds of repeat testing and delays in diagnosis. In fact, studies have shown that it can take up to 22 months from initial onset of symptoms to the diagnosis of cholangiocarcinoma, and more than one-third of patients are initially misdiagnosed with another type of cancer. To address this unmet need, the embodiments herein improve endoscopic diagnosis of indeterminate biliary strictures by risk stratifying patients that are more likely to have malignant strictures. In some embodiments, the liquid biopsy approaches described herein complement other diagnostic modalities.

The present disclosure provides a group of diagnostic and/or prognostic biomarkers (e.g., microbiomic biomarkers, metabolomic biomarkers, glycomic biomarkers, etc.) usable for diagnosis and/or assessing the relative risk of cancer in a subject. In particular, the methods and biomarkers herein find use in the diagnosis or assessment of pancreaticobiliary cancers, such as solid tumor cancers of the pancreas gland, pancreas duct, ampulla (biliary and pancreatic), bile ducts, gallbladder, liver parenchyma, duodenum, etc. In some embodiments, subsets of the biomarkers herein find use in biomarker panels of sufficient number of biomarkers to reliably diagnose and/or assess the risk of cancer in a subject but a small enough number to manageably access (e.g., 2 biomarkers, 3 biomarkers, 4 biomarkers, 5 biomarkers, 6 biomarkers, 7 biomarkers, 8 biomarkers, 9 biomarkers, 10 biomarkers, 11 biomarkers, 12 biomarkers, 13 biomarkers, 14 biomarkers, 15 biomarkers, or more biomarkers). Examples of cancers associated with pancreaticobiliary tissues and organs include, but are not limited to, pancreatic ductal adenocarcinoma, pancreatic intra-epithelial neoplasia (PanIN) lesions, intraductal papillary mucinous neoplasms (and possibly mucinous cystadenoma), cholangiocarcinoma (bile duct cancer) (with or without underlying chronic bile duct disease, specifically primary sclerosing cholangitis), hepatocellular cancer (primary liver cancer) (with or without underlying chronic liver disease, specifically cirrhosis), ampullary cancer, duodenal small bowel cancer, and gallbladder cancer.

Experiments conducted during development of embodiments herein identified diagnostic and prognostic biomarkers (e.g., microbiomic biomarkers, metabolomic biomarkers, glycomic biomarkers, etc.) that are present in different levels in subjects suffering from (or likely to suffer from) pancreaticobiliary cancers versus subjects with a benign or non-cancerous stricture, growth, or non-disease pancreaticobiliary condition. In some embodiments, analysis of such biomarkers in a sample (e.g., bile sample) from a subject provides a diagnosis, prognosis, and or risk assessment for the subject. In some embodiments, analysis of the biomarkers herein indicates a treatment courses of action that should be taken on behalf of the subject. In some embodiments, a panel of biomarkers is provided, wherein each individual biomarker is indicative of the risk/diagnosis/prognosis, but together provide an enhanced determination as to the risk/diagnosis/prognosis for the subject. In some embodiments, methods are provided for assessing the levels of the various biomarkers herein (e.g., microbiomic biomarkers, metabolomic biomarkers, glycomic biomarkers, etc.). In some embodiments, the levels of one or more biomarkers herein are compared to controls and/or threshold values. In some embodiments, methods are provided for analyzing the levels of multiple biomarkers and providing a diagnosis, prognosis, risk stratification, and/or a treatment course of action based thereupon. In some embodiments, a score is provided based on the levels of one or more biomarkers. In some embodiments, the score indicates diagnosis, prognosis, risk stratification, etc. In some embodiments, methods of treating a disease or condition identified by the biomarkers herein are provided.

In an exemplary embodiment, during ERCP, a bile sample is aspirated using the sphincterotome after deep bile cannulation into a sterile syringe. This sample can then be snap frozen in liquid nitrogen or directly processed for metabolite/microbe/glycan extraction, analysis, and/or quantification. In typical embodiments, whether the sample is identified as being indicative of pancreaticobiliary cancer, a healthy individual, or a noncancerous pancreaticobiliary condition is dependent on, for example, bacterial DNA signatures, metabolite markers, glycomic markers, and/or combinations thereof. Increases and/or decreases in various biomarkers identified in the experiments conducted during development of embodiments herein each contribute to a net determination. Experiments conducted during development of embodiments herein have identified bacterial DNA signatures upregulated in pancreaticobiliary cancers relative to noncancerous pancreaticobiliary conditions or healthy individuals (e.g., Peptostreptococcus, Neisseria, Absconditabacteria (SR1), Leptotrichia, Gemella, Lachnospiraceae, Lachnoanaerobaculum, Moigibacterium, etc.), bacterial DNA signatures downregulated in pancreaticobiliary cancers relative to noncancerous pancreaticobiliary conditions or healthy individuals (e.g., Megasphaera, Comamonadaceae, Enhydrobacter, Escherichia-Shigella, Enterococcus, Streptococcus, Actinomyces, Veillonella, etc.), metabolite signatures upregulated in pancreaticobiliary cancers relative to noncancerous pancreaticobiliary conditions or healthy individuals (e.g., urea, norepinephrine, pyroglutamic acid, N-acetyl-DL-alanine, levulinic acid, uracil, hydroxyphenyllactic, 4-trimethylammoniobutanoate, ornithine, dihydroorotate, GalNAC/Glc/NAC/ManNAC, taurine, fructose mannose, AICA ribonucleotide, carbamoyl phosphate, 2-keto-isovaleric acid, N-acetyle-DL-serine, galactosamine/glucosamine, aconitic acid, mesaconic acid, itaconic acid, b-alanine, cystosine, lysine, citrate, methylguanine, phenethelamine, 3,4-Dihydroxyphenylacetic acid, D-galactonic acid, carnitine, quinolinic acid, orotic acid, lactic acid, dehydroascorbic acid, xanthine, hypoxanthine, guanine, etc.), metabolite signatures downregulated in pancreaticobiliary cancers relative to noncancerous pancreaticobiliary conditions or healthy individuals (e.g., glycine, 3-methoxytyramine, DL-DOPA, pantothenic acid, succinic acid, 3-aminoisobutanoate, y-glutamylcysteine, alpha-ketoglutarate, 3-phosphonatooxypyruvate, glyceric acid, alanine, creatine, etc.), glycosylation and glycan markers upregulated in pancreaticobiliary cancers relative to noncancerous pancreaticobiliary conditions or healthy individuals (e.g., HexNAc4Hex4Fuc4, HexNAc5Hex3Fuc2, HexNAc3Hex2Fuc3, etc.), etc.

The metabolites of Table 1 were further identified as being significantly altered (e.g., upregulated) in the bile of subjects suffering from cancer (e.g., cholangiocarcinoma) when compared to healthy subjects and/or subjects suffering from non-cancerous conditions, such as structures. Experiments conducted during development of embodiments herein demonstrate that 3-/7-methylguanine, sucrose, 5-methylcytosine, tryptamine, N-methyltryptamine, 1-phenylethylamine, pyruvic acid, deoxyadenosine, deoxycytidine triphosphate (dCTP), picolinic acid, guanidinoacetate, NADPH, 5′methylthioadenosine, deoxyuridine, D-sedoheptulose-7-phoisphate, aspartic acid, lactose/maltose, uric acid, phenylalanine, glutamate, uridine monophosphate (UMP), uracil, and xanthine are all altered (e.g., upregulated) in the bile of subjects suffering from cancer (e.g., cholangiocarcinoma) when compared to the bile of healthy subjects or those suffering from non-cancerous conditions. Table 1 provides the fold-change in concentration of these metabolites when compared in the bile of subjects suffering from cancer (e.g., cholangiocarcinoma) when compared to the bile of healthy subjects or those suffering from non-cancerous conditions.

In various embodiments, methods are provided for diagnosing pancreaticobiliary cancer and/or other pancreaticobiliary conditions in an individual by detecting one or more biomarker values corresponding to one or more biomarkers that are present in the bile of an individual, such, by any number of analytical methods, including any of the analytical methods described herein. These biomarkers are, for example, differentially present in individuals with pancreaticobiliary cancers as compared to individuals without pancreaticobiliary cancer. Detection of the differential concentration of a biomarker in an individual can be used, for example, to permit the early diagnosis of pancreaticobiliary cancer, to distinguish between a benign and malignant mass of stricture (such as, for example, a mass observed on a computed tomography (CT) scan, MRI or ultrasound), to monitor pancreaticobiliary cancer recurrence, or for differential diagnosis from other clinical conditions such as acute or chronic pancreatitis (or both), pancreatic obstruction, GERD, gallstones, or abnormal imaging later found to be benign.

Any of the biomarkers described herein (e.g., microbiomic, metabolomic, glycomic, etc.) may be used in a variety of clinical indications for pancreaticobiliary cancer, including any of the following: detection of pancreaticobiliary cancer (e.g., pancreatic cancer); characterizing pancreaticobiliary cancer (e.g., determining cancer type, sub-type, or stage), such as by distinguishing between pancreaticobiliary cancer (e.g., pancreatic cancer, cholangiocarcinoma) and acute or chronic pancreaticobiliary conditions (e.g., pancreatitis, obstructions, GERD, autoimmune pancreatitis and cholangitis, primary sclerosing cholangitis, choledocholithiasis, or abnormal imaging later found to be benign and/or between adenocarcinoma and other malignant cell types (or otherwise facilitating histopathology); determining whether a pancreatic mass or stricture is benign or malignant; determining whether a liver mass or intrahepatic biliary stricture is benign or malignant, determining pancreaticobiliary cancer (e.g., pancreatic cancer, cholangiocarcinoma, liver cancer) prognosis; monitoring pancreaticobiliary cancer progression or remission; monitoring for pancreaticobiliary cancer recurrence; monitoring metastasis; treatment selection; monitoring response to a therapeutic agent or other treatment; stratification of individuals for endoscopic ultrasound (EUS) screening (e.g., identifying those individuals at greater risk of pancreaticobiliary cancer and thereby most likely to benefit from endosonographic biopsy, thus increasing the positive predictive value of EUS); combining biomarker testing with additional biomedical information (e.g., to provide an assay with increased diagnostic performance); facilitating the diagnosis of an abdominal mass or biliary stricture as malignant or benign; facilitating clinical decision making once an abdominal mass is observed on CT, MRI, PET or EUS (e.g., ordering repeat radiologic scans if the abdominal mass is deemed to be low risk, such as if a biomarker-based test is negative, or considering biopsy if the mass is deemed medium to high risk, such as if a biomarker-based test is positive); and facilitating decisions regarding clinical follow-up (e.g., whether to implement repeat radiologic imaging scans, fine needle biopsy, or surgery after observing an abdominal mass on imaging). The described biomarkers may also be useful in permitting diagnosis before indications of pancreaticobiliary cancer are detected by imaging modalities or other clinical correlates, or before symptoms appear. Embodiments further includes distinguishing acute or chronic pancreatitis (or both), pancreatic obstruction, GERD, gallstones, or abnormal imaging later found to be benign from pancreaticobiliary cancer.

As an example of the manner in which any of the biomarkers described herein can be used to diagnose pancreatic cancer, differential concentration of one or more of the described biomarkers in an individual who is not known to have pancreaticobiliary cancer may indicate that the individual has pancreaticobiliary cancer, thereby enabling detection of pancreaticobiliary cancer at an early stage of the disease when treatment is most effective, perhaps before the pancreaticobiliary cancer is detected by other means or before symptoms appear. Alteration (e.g., upregulation or downregulation) of one or more of the biomarkers during the course of pancreaticobiliary cancer may be indicative of pancreaticobiliary cancer progression, e.g., a pancreatic tumor is growing and/or metastasizing (and thus indicate a poor prognosis), whereas a decrease in the degree to which one or more of the biomarkers is differentially present (i.e., in subsequent biomarker tests, the expression level in the individual is moving toward or approaching a “normal” level) may be indicative of pancreaticobiliary cancer remission, e.g., a liver, bile duct or pancreatic tumor is shrinking (and thus indicate a good or better prognosis). Similarly, an increase in the degree to which one or more of the biomarkers is differentially expressed (i.e., in subsequent biomarker tests, the expression level in the individual is moving further away from a “normal” level) during the course of pancreaticobiliary cancer treatment may indicate that the pancreaticobiliary cancer is progressing and therefore indicate that the treatment is ineffective, whereas a decrease in differential expression of one or more of the biomarkers during the course of pancreaticobiliary cancer treatment may be indicative of pancreaticobiliary cancer remission and therefore indicate that the treatment is working successfully. Additionally, an increase or decrease in the differential concentration of one or more of the biomarkers after an individual has apparently been cured of pancreaticobiliary cancer may be indicative of pancreatic cancer recurrence.

Detection of any of the biomarkers described herein may be particularly useful following, or in conjunction with, pancreaticobiliary cancer treatment, such as to evaluate the success of the treatment or to monitor pancreaticobiliary cancer remission, recurrence, and/or progression (including metastasis) following treatment. pancreaticobiliary cancer treatment may include, for example, administration of a therapeutic agent to the individual, performance of surgery (e.g., surgical resection of at least a portion of a pancreaticobiliary tumor or removal of pancreaticobiliary (e.g., pancreatic) and surrounding tissue), administration of radiation therapy, or any other type of pancreatic cancer treatment used in the art, and any combination of these treatments. For example, any of the biomarkers may be detected at least once after treatment or may be detected multiple times after treatment (such as at periodic intervals), or may be detected both before and after treatment. Differential levels of any of the biomarkers in an individual over time may be indicative of pancreatic cancer progression, remission, or recurrence, etc.

In some embodiments, a biological sample is obtained from a subject. In some embodiments, the biological sample is tested for biomarkers (e.g., microbiomic, metabolomic, glycomic, etc.), and an analysis is performed to provide a clinician with a prognosis and treatment course of action. In some embodiments, a biological sample is a bile sample. In some embodiments, a bile sample and/or liquid biopsy is obtained by/during endoscopic retrograde cholangiopancreatography (ERCP) or another suitable technique or procedure (surgical or interventional radiology). In some embodiments, a sample is a fluid biopsy sample. For example, during ERCP, one or more (e.g., 1, 2, 3, 4, or more) bile samples (e.g., 0.1-5.0 ml samples (e.g., 0.5 ml, 1.0 ml, 1.5 ml, 2.0, ml, 2.5 ml, 3.0 ml, 3.5 ml, 4.0 ml, 4.5 ml, 5.0 ml, or ranges therebetween) are aspirated (e.g., using a sphincterotome) after deep bile cannulation into a sterile syringe. Other methods of obtaining a bile sample include direct aspiration of the gallbladder, by duodenal aspirate, percutaneous biliary drain, or by T-tube drainage. Methods herein are not limited by the technique used for obtaining able sample. In some embodiments, liquid bile sample is obtained while obtaining cells/tissues (e.g., solid biopsy) for separate analysis (e.g., cytology, histology, etc.),

In some embodiments, a sample can then be snap frozen in liquid nitrogen or directly processed for metabolite extraction. Both polar and non-polar metabolites are extracted and then submitted for quantification using liquid chromatography and mass spectrometry. Whether the sample is identified as benign or malignant is dependent on the combination of bacterial DNA signatures and metabolite markers.

In some embodiments, provided herein are species or taxonomic groups of microbes (e.g., bacteria), the presence/absence or level of which is a subject or sample from the subject is diagnostic and/or prognostic of the relative likelihood of pancreaticobiliary cancers versus subjects with a benign or non-cancerous stricture, growth, or non-disease pancreaticobiliary condition. In some embodiments, provided herein are panels of such microbiomic biomarkers.

In some embodiments, an increased level (e.g., above a threshold abundance) of one or more bacterial species (e.g., from the genera Peptostreptococcus, Neisseria, Absconditabacteria (SR1), Leptotrichia, Gemella, Lachnospiraceae, Lachnoanaerobaculum, and/or Moigibacterium) is associated with increased risk of one or more pancreaticobiliary cancers. In some embodiments, a decreased level (e.g., below a threshold abundance) of the one or more bacterial species (e.g., from the genera Peptostreptococcus, Neisseria, Absconditabacteria (SR1), Leptotrichia, Gemella, Lachnospiraceae, Lachnoanaerobaculum, and/or Moigibacterium) is associated with a decreased risk of one or more pancreaticobiliary cancers. In some embodiments, bacteria of one or more of the following genera is upregulated (e.g., increased in abundance) in subjects with a malignant pancreaticobiliary stricture (e.g., bile duct stricture, pancreatic stricture, etc.), pancreaticobiliary cancer, pancreaticobiliary tumor, etc. compared to a healthy subject and/or a subject suffering from a non-cancerous/benign pancreaticobiliary conditions: Peptostreptococcus, Neisseria, Absconditabacteria (SR1), Leptotrichia, Gemella, Lachnospiraceae, Lachnoanaerobaculum, and Moigibacterium. In some embodiments, bacteria of one or more of the following genera is downregulated (e.g., decreased in abundance) in a healthy subject and/or a subject suffering from a non-cancerous/benign pancreaticobiliary condition compared to a subject with a malignant pancreaticobiliary stricture (e.g., bile duct stricture, pancreatic stricture, etc.), pancreaticobiliary cancer, pancreaticobiliary tumor, etc.: Peptostreptococcus, Neisseria, Absconditabacteria (SR1), Leptotrichia, Gemella, Lachnospiraceae, Lachnoanaerobaculum, and Moigibacterium.

In some embodiments, a decreased level (e.g., below a threshold abundance) of one or more bacterial species (e.g., from the genera Megasphaera, Comamonadaceae, Enhydrobacter, Escherichia-Shigella, Enterococcus, Streptococcus, Actinomyces, Veillonella, etc.) is associated with increased risk of one or more pancreaticobiliary cancers. In some embodiments, an increased level (e.g., above a threshold abundance) of the one or more bacterial species (e.g., from the genera Megasphaera, Comamonadaceae, Enhydrobacter, Escherichia-Shigella, Enterococcus, Streptococcus, Actinomyces, Veillonella, etc.) is associated with a decreased risk of one or more pancreaticobiliary cancers. In some embodiments, bacteria of one or more of the following genera is downregulated (e.g., decreased in abundance) in subjects with a malignant pancreaticobiliary stricture (e.g., bile duct stricture, pancreatic stricture, etc.), pancreaticobiliary cancer, pancreaticobiliary tumor, etc. compared to a healthy subject and/or a subject suffering from a non-cancerous/benign pancreaticobiliary conditions: Megasphaera, Comamonadaceae, Enhydrobacter, Escherichia-Shigella, Enterococcus, Streptococcus, Actinomyces, and/or Veillonella. In some embodiments, bacteria of one or more of the following genera is upregulated (e.g., increased in abundance) in a healthy subject and/or a subject suffering from a non-cancerous/benign pancreaticobiliary condition compared to a subject with a malignant pancreaticobiliary stricture (e.g., bile duct stricture, pancreatic stricture, etc.), pancreaticobiliary cancer, pancreaticobiliary tumor, etc.: Megasphaera, Comamonadaceae, Enhydrobacter, Escherichia-Shigella, Enterococcus, Streptococcus, Actinomyces, and/or Veillonella.

In some embodiments, microbiomic biomarkers are assessed (e.g., quantified) is a sample form a subject by any suitable method. In some embodiments, a sample is tested to determine the microbiomic makeup of the sample. In some embodiments, specific species or taxonomic groups (e.g., genus) of bacteria are tested for (e.g., species of taxa that are diagnostic/prognostic of risk of one or more pancreaticobiliary cancers), for example: Peptostreptococcus, Neisseria, Absconditabacteria (SR1), Leptotrichia, Gemella, Lachnospiraceae, Lachnoanaerobaculum, moigibacterium, Megasphaera, Comamonadaceae, Enhydrobacter, Escherichia-Shigella, Enterococcus, Streptococcus, Actinomyces, and Veillonella. In some embodiments, intact bacteria are detected (e.g., by detecting surface polypeptides or markers). In other embodiments, bacteria are lysed and nucleic acids or proteins (e.g., corresponding to 16S rRNA or genes specific to the species of bacteria) are detected. In some embodiments, bacteria are identified using detection reagents (e.g., a probe, a microarray, e.g., an amplification primer) that specifically interact with a nucleic acid that identifies a particular species or taxa of bacteria.

Some embodiments comprise use of nucleic acid sequencing to detect, quantify, and/or identify gut microbiota. The term “sequencing,” as used herein, refers to a method by which the identity of at least 10 consecutive nucleotides (e.g., the identity of at least 20, at least 50, at least 100, or at least 200 or more consecutive nucleotides) of a polynucleotide are obtained. The term “next-generation sequencing” refers to the so-called parallelized sequencing-by-synthesis, sequencing-by-ligation platforms, nanopore sequencing methods, or electronic-detection based methods that will be understood in the field.

Some embodiments comprise acquiring a bile microbiota sample from a subject. Methods for obtaining a bile sample from a subject are described herein. Suitable bile samples may be freshly obtained or may have been stored under appropriate temperatures (e.g., frozen). Methods for extracting nucleic acids from a bile sample are provided. The extracted nucleic acids may or may not be amplified prior to being used as an input for profiling the relative abundances of bacterial taxa, depending upon the type and sensitivity of the downstream method. When amplification is desired, nucleic acids may be amplified via polymerase chain reaction (PCR). Methods for performing PCR are well known in the art. Selection of nucleic acids or regions of nucleic acids to amplify are discussed above. The nucleic acids comprising the nucleic acid sample may also be fluorescently or chemically labeled, fragmented, or otherwise modified prior to sequencing or hybridization to an array as is routinely performed in the art.

In some embodiments, nucleic acids are amplified using primers that are compatible with use in, e.g., Illumina's reversible terminator method, Roche's pyrosequencing method (454), Life Technologies's sequencing by ligation (the SOLiD platform) or Life Technologies's Ion Torrent platform. Examples of such methods are described in the following references: Margulies et al (Nature 2005 437: 376-80); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol Biol. 2009; 553:79-108); Appleby et al (Methods Mol Biol. 2009; 513: 19-39) and Morozova et al (Genomics. 2008 92:255-64), which are incorporated by reference for the general descriptions of the methods and the particular steps of the methods, including all starting products, reagents, and final products for each of the steps.

In another embodiment, the isolated microbial DNA may be sequenced using nanopore sequencing (e.g., as described in Soni et al. Clin Chem 2007 53: 1996-2001, or as described by Oxford Nanopore Technologies). Nanopore sequencing technology is disclosed in U.S. Pat. Nos. 5,795,782, 6,015,714, 6,627,067, 7,238,485 and 7,258,838 and U.S. Pat Appln Nos. 2006003171 and 20090029477.

The isolated microbial fragments may be sequenced directly or, in some embodiments, the isolated microbial fragments may be amplified (e.g., by PCR) to produce amplification products that sequenced. In certain embodiments, amplification products may contain sequences that are compatible with use in, e.g., Illumina's reversible terminator method, Roche's pyrosequencing method (454), Life Technologies' sequencing by ligation (the SOLiD platform) or Life Technologies' Ion Torrent platform, as described above. Embodiments herein are not limited by the techniques used to identify and/or quantify the bacteria present in a sample.

In some embodiments, provided herein are species or taxonomic groups of microbes (e.g., bacteria), the presence/absence or level of which is a subject or sample from the subject is diagnostic and/or prognostic of pancreaticobiliary cancer. In some embodiments, provided herein are panels of such microbiomic biomarkers.

Experiments conducted during development of embodiments herein demonstrate that the levels of various small molecule metabolites present in biological samples (e.g., bile) from a subject correlate with and are diagnostic/prognostic of the risk of pancreaticobiliary cancer for the subject. In some embodiments, provided herein are panels of metabolites, the levels of which in biological samples (e.g., bile, etc.) from a subject correlate with the risk of the subject suffering from one or more pancreaticobiliary cancers. In some embodiments, methods are provided of assessing the levels of such metabolomic biomarkers in biological samples (e.g., bile, etc.) from a subject.

In some embodiments, an increased level (e.g., above a threshold abundance) of one or more metabolites (e.g., urea, norepinephrine, pyroglutamic acid, N-acetyl-DL-alanine, levulinic acid, uracil, hydroxyphenyllactic, 4-trimethylammoniobutanoate, ornithine, dihydroorotate, GalNAC/Glc/NAC/ManNAC, taurine, fructose mannose, AICA ribonucleotide, carbamoyl phosphate, 2-keto-isovaleric acid, N-acetyle-DL-serine, galactosamine/glucosamine, aconitic acid, mesaconic acid, itaconic acid, b-alanine, cystosine, lysine, citrate, methylguanine, phenethelamine, 3,4-Dihydroxyphenylacetic acid, D-galactonic acid, carnitine, quinolinic acid, orotic acid, lactic acid, dehydroascorbic acid, xanthine, hypoxanthine, guanine, etc.) is associated with increased risk of one or more pancreaticobiliary cancers. In some embodiments, a decreased level (e.g., below a threshold abundance) of the one or more metabolites (e.g., urea, norepinephrine, pyroglutamic acid, N-acetyl-DL-alanine, levulinic acid, uracil, hydroxyphenyllactic, 4-trimethylammoniobutanoate, ornithine, dihydroorotate, GalNAC/Glc/NAC/ManNAC, taurine, fructose mannose, AICA ribonucleotide, carbamoyl phosphate, 2-keto-isovaleric acid, N-acetyle-DL-serine, galactosamine/glucosamine, aconitic acid, mesaconic acid, itaconic acid, b-alanine, cystosine, lysine, citrate, methylguanine, phenethelamine, 3,4-Dihydroxyphenylacetic acid, D-galactonic acid, carnitine, quinolinic acid, orotic acid, lactic acid, dehydroascorbic acid, xanthine, hypoxanthine, guanine, etc.) is associated with a decreased risk of one or more pancreaticobiliary cancers. In some embodiments, one or more of the following metabolites is upregulated (e.g., increased in abundance) in subjects with a malignant pancreaticobiliary stricture (e.g., bile duct stricture, pancreatic stricture, etc.), pancreaticobiliary cancer, pancreaticobiliary tumor, etc. compared to a healthy subject and/or a subject suffering from a non-cancerous/benign pancreaticobiliary conditions: urea, norepinephrine, pyroglutamic acid, N-acetyl-DL-alanine, levulinic acid, uracil, hydroxyphenyllactic, 4-trimethylammoniobutanoate, ornithine, dihydroorotate, GalNAC/Glc/NAC/ManNAC, taurine, fructose mannose, AICA ribonucleotide, carbamoyl phosphate, 2-keto-isovaleric acid, N-acetyle-DL-serine, galactosamine/glucosamine, aconitic acid, mesaconic acid, itaconic acid, b-alanine, cystosine, lysine, citrate, methylguanine, phenethelamine, 3,4-Dihydroxyphenylacetic acid, D-galactonic acid, carnitine, quinolinic acid, orotic acid, lactic acid, dehydroascorbic acid, xanthine, hypoxanthine, and/or guanine. In some embodiments, one or more of the following metaboilites is downregulated (e.g., decreased in abundance) in a healthy subject and/or a subject suffering from a non-cancerous/benign pancreaticobiliary condition compared to a subject with a malignant pancreaticobiliary stricture (e.g., bile duct stricture, pancreatic stricture, etc.), pancreaticobiliary cancer, pancreaticobiliary tumor, etc.: urea, norepinephrine, pyroglutamic acid, N-acetyl-DL-alanine, levulinic acid, uracil, hydroxyphenyllactic, 4-trimethylammoniobutanoate, ornithine, dihydroorotate, GalNAC/Glc/NAC/ManNAC, taurine, fructose mannose, AICA ribonucleotide, carbamoyl phosphate, 2-keto-isovaleric acid, N-acetyle-DL-serine, galactosamine/glucosamine, aconitic acid, mesaconic acid, itaconic acid, b-alanine, cystosine, lysine, citrate, methylguanine, phenethelamine, 3,4-Dihydroxyphenylacetic acid, D-galactonic acid, carnitine, quinolinic acid, orotic acid, lactic acid, dehydroascorbic acid, xanthine, hypoxanthine, and/or guanine.

In some embodiments, a decreased level (e.g., below a threshold abundance) of one or more metabolites (e.g., glycine, 3-methoxytyramine, DL-DOPA, pantothenic acid, succinic acid, 3-aminoisobutanoate, y-glutamylcysteine, alpha-ketoglutarate, 3-phosphonatooxypyruvate, glyceric acid, alanine, creatine, etc.) is associated with increased risk of one or more pancreaticobiliary cancers. In some embodiments, an increased level (e.g., above a threshold abundance) of the one or more metaboilites (e.g., glycine, 3-methoxytyramine, DL-DOPA, pantothenic acid, succinic acid, 3-aminoisobutanoate, y-glutamylcysteine, alpha-ketoglutarate, 3-phosphonatooxypyruvate, glyceric acid, alanine, creatine, etc.) is associated with a decreased risk of one or more pancreaticobiliary cancers. In some embodiments, one or more of the following metabolites is downregulated (e.g., decreased in abundance) in subjects with a malignant pancreaticobiliary stricture (e.g., bile duct stricture, pancreatic stricture, etc.), pancreaticobiliary cancer, pancreaticobiliary tumor, etc. compared to a healthy subject and/or a subject suffering from a non-cancerous/benign pancreaticobiliary conditions: glycine, 3-methoxytyramine, DL-DOPA, pantothenic acid, succinic acid, 3-aminoisobutanoate, y-glutamylcysteine, alpha-ketoglutarate, 3-phosphonatooxypyruvate, glyceric acid, alanine, and/or creatine. In some embodiments, bacteria of one or more of the following genera is upregulated (e.g., increased in abundance) in a healthy subject and/or a subject suffering from a non-cancerous/benign pancreaticobiliary condition compared to a subject with a malignant pancreaticobiliary stricture (e.g., bile duct stricture, pancreatic stricture, etc.), pancreaticobiliary cancer, pancreaticobiliary tumor, etc.: glycine, 3-methoxytyramine, DL-DOPA, pantothenic acid, succinic acid, 3-aminoisobutanoate, y-glutamylcysteine, alpha-ketoglutarate, 3-phosphonatooxypyruvate, glyceric acid, alanine, and/or creatine.

In some embodiments, an altered level (e.g., increased) of one or more metabolites of Table 1 in the bile of a subject is indicative of cancer (e.g., cholangiocarcinoma) rather than non-cancerous conditions, such as structures. Experiments conducted during development of embodiments herein demonstrate that 3-/7-methylguanine, sucrose, 5-methylcytosine, tryptamine, N-methyltryptamine, 1-phenylethylamine, pyruvic acid, deoxyadenosine, deoxycytidine triphosphate (dCTP), picolinic acid, guanidinoacetate, NADPH, 5′methylthioadenosine, deoxyuridine, D-sedoheptulose-7-phoisphate, aspartic acid, lactose/maltose, uric acid, phenylalanine, glutamate, uridine monophosphate (UMP), uracil, and xanthine are all altered (e.g., upregulated) in the bile of subjects suffering from cancer (e.g., cholangiocarcinoma) when compared to the bile of healthy subjects or those suffering from non-cancerous conditions. Table 1 provides the fold-change in concentration of these metabolites when compared in the bile of subjects suffering from cancer (e.g., cholangiocarcinoma) when compared to the bile of healthy subjects or those suffering from non-cancerous conditions.

In some embodiments, any technique and/or instrumentation suitable for detecting/quantifying small molecule metabolites in a complex environment may find use in embodiments herein. In some embodiments, analytical platforms (e.g., High-throughput platforms, automated platforms, etc.) utilizing nuclear magnetic resonance (NMR) spectroscopy, gas chromatography (GC), liquid chromatography (LC), and/or mass spectrometry (MS) are employed to measure the metabolites within a biological sample. In some embodiments, NMR, GC, and/or LC coupled to MS is utilized.

Mass spectrometry can accurately identify/quantify thousands of metabolites within complex biological samples. In some embodiments, metabolites are detected/quantified in a biological sample using MS techniques, such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, tandem mass spectrometry (e.g., MS/MS, MS/MS/MS, ESI-MS/MS etc.), secondary ion mass spectrometry (SIMS), or ion mobility spectrometry (e.g. GC-IMS, IMS-MS, LC-IMS, LC-IMS-MS etc.). Mass spectrometry methods are well known in the art and have been used to quantify and/or identify biomolecules, such metabolites.

In certain embodiments, a gas phase ion spectrophotometer is used. In other embodiments, laser-desorption/ionization mass spectrometry is used to identify metabolites. Modem laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the metabolite is mixed with a solution containing a matrix, and a drop of the liquid is placed on the surface of a substrate. The matrix solution then co-crystallizes with the biomarkers. The substrate is inserted into the mass spectrometer. Laser energy is directed to the substrate surface where it desorbs and ionizes the proteins without significantly fragmenting them. However, MALDI has limitations as an analytical tool. It does not provide means for fractionating the biological fluid, and the matrix material can interfere with detection, especially for low molecular weight analytes. In SELDI, the substrate surface is modified so that it is an active participant in the desorption process. In one variant, the surface is derivatized with adsorbent and/or capture reagents that selectively bind the biomarker of interest. In another variant, the surface is derivatized with energy absorbing molecules that are not desorbed when struck with the laser. In another variant, the surface is derivatized with molecules that bind the biomarker of interest and that contain a photolytic bond that is broken upon application of the laser. In each of these methods, the derivatizing agent generally is localized to a specific location on the substrate surface where the sample is applied. The two methods can be combined by, for example, using a SELDI affinity surface to capture an analyte (e.g. biomarker) and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094; incorporated by reference in their entireties.

In some embodiments, the data from mass spectrometry is represented as a mass chromatogram. A “mass chromatogram” is a representation of mass spectrometry data as a chromatogram. Typically, the x-axis represents time and the y-axis represents signal intensity. In one aspect the mass chromatogram is a total ion current (TIC) chromatogram. In another aspect, the mass chromatogram is a base peak chromatogram. In other embodiments, the mass chromatogram is a selected ion monitoring (SIM) chromatogram. In yet another embodiment, the mass chromatogram is a selected reaction monitoring (SRM) chromatogram. In one embodiment, the mass chromatogram is an extracted ion chromatogram (EIC). In an EIC, a single feature is monitored throughout the entire run. The total intensity or base peak intensity within a mass tolerance window around a particular analyte's mass-to-charge ratio is plotted at every point in the analysis. The size of the mass tolerance window typically depends on the mass accuracy and mass resolution of the instrument collecting the data. As used herein, the term “feature” refers to a single small metabolite, or a fragment of a metabolite. In some embodiments, the term feature may also include noise upon further investigation.

In some embodiments, detection of the presence of a metabolite involves detection of signal intensity. This, in turn, can reflect the quantity and character of a biomarker. For example, in certain embodiments, the signal strength of peak values from spectra of a first sample and a second sample can be compared (e.g., visually, by computer analysis etc.) to determine the relative amounts of particular metabolites. Software programs such as the Biomarker Wizard program (Ciphergen Biosystems, Inc., Fremont, Calif.) can be used to aid in analyzing mass spectra. The mass spectrometers and their techniques are well known.

A person skilled in the art understands that any of the components of a mass spectrometer, e.g., desorption source, mass analyzer, detect, etc., and varied sample preparations can be combined with other suitable components or preparations described herein, or to those known in the art. For example, in some embodiments a control sample may contain heavy atoms, e.g. ¹³C, thereby permitting the test sample to be mixed with the known control sample in the same mass spectrometry run.

In some embodiments, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio. In one embodiment of the invention, levels of metabolites are detected by MALDI-TOF mass spectrometry.

Methods of detecting metabolites also include the use of surface plasmon resonance (SPR). The SPR biosensing technology has been combined with MALDI-TOF mass spectrometry for the desorption and identification of metabolites.

Data for statistical analysis can be extracted from chromatograms (spectra of mass signals) using software for statistical methods known in the art. “Statistics” is the science of making effective use of numerical data relating to groups of individuals or experiments. Methods for statistical analysis are well-known in the art. In one embodiment a computer is used for statistical analysis. In one embodiment, the Agilent MassProfiler or MassProfilerProfessional software is used for statistical analysis. In another embodiment, the Agilent MassHunter software Qual software is used for statistical analysis. In other embodiments, alternative statistical analysis methods can be used. Such other statistical methods include the Analysis of Variance (ANOVA) test, Chi-square test, Correlation test, Factor analysis test, Mann-Whitney U test, Mean square weighted derivation (MSWD), Pearson product-moment correlation coefficient, Regression analysis, Spearman's rank correlation coefficient, Student's T test, Welch's T-test, Tukey's test, and Time series analysis.

In various embodiments, signals from mass spectrometry are transformed in different ways to improve the performance of the method. Either individual signals or summaries of the distributions of signals (such as mean, median or variance) can be so transformed. Possible transformations include taking the logarithm, taking some positive or negative power, for example the square root or inverse, or taking the arcsin (Myers, Classical and Modern Regression with Applications, 2nd edition, Duxbury Press, 1990).

In some embodiments, the ability to quantitate the amount of a metabolite (or multiple metabolite) in a biological sample from a subject allows a clinician to make assessments regrading the condition of the subject, to further provide a diagnosis or prognosis for the subject, and/or to further recommend or administer a treatment course of action. Thus, according to another aspect of the present invention there is provided a method of characterizing the risk of one or more pancreaticobiliary cancers comprising determining the abundance of at least one metabolite in a biological sample from the subject (e.g., bile, etc.), comparing the quantity to a control or threshold value, and providing a prognosis regrading the risk of one or more pancreaticobiliary cancers and/or a treatment course of action for the subject.

Once the level of one or more metabolites is measured by the techniques described herein and/or understood in the field, it is typically compared to a level of that metabolite in a control subject or a threshold value. In some embodiments, if the metabolite level is above or below the control or threshold, determinations about the state or the subject can be inferred or concluded. In embodiments in which the levels of multiple metabolites are measured to provide a prognosis or to determine a treatment course of action, an algorithm may be employed to combine the level of the multiple biomarkers, and/or their levels relative to individual thresholds, into a single prognosis or treatment course of action. In some embodiments, a score is provided for each metabolite, based on the abundance of the metabolite in the sample relative to a control or threshold value. In some embodiments, the combination of scores from multiple metabolites is used to provide a prognosis and/or determine a treatment course of action.

In some embodiments, one or more glycosylation and glycan markers are detected and/or quantitated as biomarkers within the scope herein. In some embodiments, glycosylation and glycan biomarkers useful in the embodiments described herein include but are not limited to: HexNAc₄Hex₄Fuc₃, HexNAc4Hex5NeuAc1, HexNAc4Hex4Fuc4, HexNAc5Hex4Fuc3, HexNAc4Hex5NeuAc2, HexNAc5Hex4Fuc4, HexNAc3Hex3Fuc2, HexNAc4Hex2Fuc2, HexNAc4Hex3Fuc1, HexNAc4Hex4, HexNAc5Hex3, HexNAc3Hex2NeuAc1Fuc2, HexNAc3Hex3Fuc3, HexNAc3Hex3NeuAc1Fuc1, HexNAc2Hex4Fuc3, HexNAc4Hex3Fuc2, HexNAc4Hex4Fuc1, HexNAc5Hex3Fuc1, HexNAc3Hex3Fuc4, HexNAc3Hex3NeuAc1Fuc2, HexNAc4Hex3Fuc3, HexNAc4Hex4Fuc2, HexNAc5Hex3Fuc2, HexNAc5Hex4Fuc1, HexNAc4Hex3Fuc4, HexNAc4Hex3NeuAc1Fuc2, HexNAc2Hex2NeuAc1, HexNAc2Hex3Fuc1, HexNAc3Hex2Fuc1, HexNAc3Hex3Fuc1, HexNAc4Hex2, HexNAc2Hex2Fuc3, HexNAc2Hex2NeuAc1Fuc1, HexNAc2Hex3Fuc2, HexNAc3Hex2Fuc2, HexNAc3Hex2NeuAc1, HexNAc3Hex3Fuc1, HexNAc1Hex3NeuAc1Fuc2, HexNAc1Hex3NeuAc1, HexNAc4Hex3, HexNAc2Hex2NeuAc1Fuc2, HexNAc2Hex2NeuAc2, HexNAc2Hex3NeuAc1Fuc1, HexNAc2Hex4Fuc2, HexNAc3Hex2Fuc3, HexNAc3Hex2NeuAc1Fuc1, HexNAc1Hex1, HexNAc2, HexNAc1NeuAc1, HexNAc1Hex1Fuc1, HexNAc2Hex1, HexNAc1Hex1NeuAc1, HexNAc2NeuAc1, HexNAc2Hex1Fuc1, HexNAc2Hex2, HexNAc3Hex1, HexNAc1Hex1NeuAc1Fuc1, HexNAc1Hex2Fuc2, HexNAc2Hex1NeuAc1, HexNAc2Hex2Fuc1, HexNAc3Hex1Fuc1, HexNAc3Hex2, HexNAc1Hex1NeuAc2, HexNAc1Hex3NeuAc1, HexNAc2Hex1NeuAc1Fuc1, HexNAc2Hex2Fuc2, (Hex)3 (HexNAc)3 (Deoxyhexose)1 (NeuAc)3+(Man)3(GlcNAc)2, (Hex)3 (HexNAc)3 (Deoxyhexose)2 (NeuAc)3+(Man)3(GlcNAc)2, (Hex)4 (HexNAc)4 (NeuAc)3+(Man)3(GlcNAc)2, (Hex)4 (HexNAc)4 (Deoxyhexose)1 (NeuAc)3+(Man)3(GlcNAc)2, (Hex)4 (HexNAc)4 (NeuAc)4+(Man)3(GlcNAc)2, (Hex)4 (HexNAc)4 (Deoxyhexose)1 (NeuAc)4+(Man)3(GlcNAc)2, (Hex)2 (HexNAc)2 (NeuAc)1 (NeuGc)1+(Man)3(GlcNAc)2, (Hex)2 (HexNAc)3 (Deoxyhexose)1 (NeuAc)1+, (Man)3(GlcNAc)2, (Hex)3 (HexNAc)3 (NeuAc)1+(Man)3(GlcNAc)2, (Hex)2 (HexNAc)4 (Deoxyhexose)2+(Man)3(GlcNAc)2, (Hex)2 (HexNAc)2 (Deoxyhexose)3 (NeuAc)1+(Man)3(GlcNAc)2, (Hex)2 (HexNAc)2 (Deoxyhexose)1 (NeuAc)2+(Man)3(GlcNAc)2, (Hex)3 (HexNAc)2 (NeuAc)2+(Man)3(GlcNAc)2 (Hex)2 (HexNAc)3 (Deoxyhexose)4+(Man)3(GlcNAc)2, (Hex)2 (HexNAc)3 (Deoxyhexose)2 (NeuAc)1+(Man)3(GlcNAc)2, (Hex)3 (HexNAc)3 (Deoxyhexose)1 (NeuAc)1+(Man)3(GlcNAc)2, (Hex)2 (HexNAc)4 (Deoxyhexose)3+(Man)3(GlcNAc)2, (Hex)2 (HexNAc)3 (Deoxyhexose)3 (NeuAc)1+(Man)3(GlcNAc)2, (Hex)2 (HexNAc)3 (Deoxyhexose)1 (NeuAc)2+(Man)3(GlcNAc)2, (Hex)3 (HexNAc)3 (NeuAc)2+(Man)3(GlcNAc)2, (Hex)3 (HexNAc)4 (Deoxyhexose)3+(Man)3(GlcNAc)2, (Hex)3 (HexNAc)3 (Deoxyhexose)1 (NeuAc)2+(Man)3(GlcNAc)2, (Hex)3 (HexNAc)4 (Deoxyhexose)4+(Man)3(GlcNAc)2, (Hex)3 (HexNAc)3 (NeuAc)3+(Man)3(GlcNAc)2, (Hex)3 (HexNAc)4 (Deoxyhexose)3 (NeuAc)1+(Man)3(GlcNAc)2 (Hex)4 (HexNAc)4 (NeuAc)2+(Man)3(GlcNAc)2, (Hex)6+(Man)3(GlcNAc)2, (Hex)1 (HexNAc)2 (Deoxyhexose)1 (NeuAc)1+(Man)3(GlcNAc)2, (Hex)2 (HexNAc)2 (NeuAc)1+(Man)3(GlcNAc)2, (Hex)1 (HexNAc)3 (Deoxyhexose)2+(Man)3(GlcNAc)2, (Hex)1 (HexNAc)3 (NeuAc)1+(Man)3(GlcNAc)2, (Hex)2 (HexNAc)3 (Deoxyhexose)1+(Man)3(GlcNAc)2, (Hex)2 (HexNAc)1 (NeuAc)2+(Man)3(GlcNAc)2, (Hex)2 (HexNAc)2 (Deoxyhexose)1 (NeuAc)1+(Man)3(GlcNAc)2, (Hex)1 (HexNAc)3 (Deoxyhexose)3+(Man)3(GlcNAc)2, (Hex)1 (HexNAc)3 (Deoxyhexose)1 (NeuAc)1+(Man)3(GlcNAc)2, (Hex)2 (HexNAc)3 (Deoxyhexose)2+(Man)3(GlcNAc)2, (Hex)2 (HexNAc)3 (NeuAc)1+(Man)3(GlcNAc)2, (Hex)5 (HexNAc)2+(Man)3(GlcNAc)2, (Hex)2 (HexNAc)1 (Deoxyhexose)1 (NeuAc)2+(Man)3(GlcNAc)2, (Hex)1 (HexNAc)5 (Deoxyhexose)5+(Man)3(GlcNAc)2, (Hex)1 (HexNAc)2 (Deoxyhexose)1 (NeuAc)2+(Man)3(GlcNAc)2, (Hex)1 (HexNAc)5 (Deoxyhexose)1+(Man)3(GlcNAc)2, (Hex)2 (HexNAc)2 (NeuAc)2+(Man)3(GlcNAc)2, (Hex)2 (HexNAc)5+(Man)3(GlcNAc)2, (Hex)3 (HexNAc)2 (Deoxyhexose)1 (NeuAc)1+(Man)3(GlcNAc)2, (Hex)2 (HexNAc)2, (Man)3(GlcNAc)2, (Hex)2+(Man)3(GlcNAc)2, (HexNAc)2+(Man)3(GlcNAc)2, (Hex)3+(Man)3(GlcNAc)2, (HexNAc)2 (Deoxyhexose)1+(Man)3(GlcNAc)2, (HexNAc)3+(Man)3(GlcNAc)2, (Hex)1 (HexNAc)1 (NeuAc)1+(Man)3(GlcNAc)2, (Hex)4+(Man)3(GlcNAc)2, (Hex)1 (HexNAc)2 (Deoxyhexose)1+(Man)3(GlcNAc)2, (Hex)2 (HexNAc)2+(Man)3(GlcNAc)2, (HexNAc)3 (Deoxyhexose)1+(Man)3(GlcNAc)2, (Hex)1 (HexNAc)3+(Man)3(GlcNAc)2, (Hex)2 (HexNAc)1 (NeuAc)1+(Man)3(GlcNAc)2, (Hex)5+(Man)3(GlcNAc)2, (Hex)1 (HexNAc)2 (Deoxyhexose)2+(Man)3(GlcNAc)2, (Hex)1 (HexNAc)2 (NeuAc)1+(Man)3(GlcNAc)2, (Hex)2 (HexNAc)2 (Deoxyhexose)1+(Man)3(GlcNAc)2, (Hex)1 (HexNAc)3 (Deoxyhexose)1+(Man)3(GlcNAc)2, and (Hex)3 (HexNAc)1 (NeuAc)1+(Man)3(GlcNAc)2. In particular embodiments, the glycosylation and glycan biomarkers HexNAc4Hex4Fuc4, HexNAc5Hex3Fuc2, and HexNAc3Hex2Fuc3 are increased in subject suffering from pancreaticobiliary cancers relative to noncancerous pancreaticobiliary conditions or healthy individuals.

In some embodiments, the biomarkers and methods described herein find use with one or more companion diagnostic techniques. In some embodiments, the biomarkers and methods described herein find use instead of one or more existing diagnostic techniques commonly used for assessing pancreaticobiliary conditions (e.g., cancer). In some embodiments, existing diagnostic techniques are used to obtain additional biomedical information to be used with or replaced by the biomarkers and methods herein. “Additional biomedical information” refers to one or more evaluations of an individual, other than using any of the biomarkers described herein, that are associated with cancer risk or, more specifically, pancreaticobiliary cancer risk, or for distinguishing pancreaticobiliary cancer from other benign or noncancerous pancreaticobiliary conditions. “Additional biomedical information” includes any of the following: physical descriptors of an individual, including a pancreatic mass observed by any of contrast-enhanced multislice (multidetector) helical computed tomography (CT) scanning with three dimensional reconstruction, transcutaneous or endoscopic ultrasound (US or EUS), endoscopic retrograde cholangiopancreatography (ERCP), magnetic resonance imaging (MRI), MR cholangiopancreatography (MRCP), or abdominal ultrasound; the height and/or weight of an individual; change in weight; the ethnicity of an individual; occupational history; family history of pancreatic cancer (or other cancer); the presence of a genetic marker(s) correlating with a higher risk of pancreatic cancer (or other cancer) in the individual or a family member; the presence or absence of a pancreatic mass or other abdominal mass; size of mass; location of mass; morphology of mass and associated abdominal region (e.g., as observed through imaging); clinical symptoms such as abdominal pain, weight loss, anorexia, early satiety, diarrhea, or steatorrhea, jaundice, recent onset of atypical diabetes mellitus, a history of recent but unexplained thrombophlebitis, or previous attack of pancreatitis, and the like; gene expression values; physical descriptors of an individual, including physical descriptors observed by radiologic imaging; the height and/or weight of an individual; the gender of an individual; the ethnicity of an individual; smoking history; alcohol use history; occupational history; exposure to known carcinogens (e.g., exposure to any of asbestos, radon gas, chemicals, smoke from fires, and air pollution, which can include emissions from stationary or mobile sources such as industrial/factory or auto/marine/aircraft emissions); exposure to second-hand smoke; and family history of pancreatic cancer or other cancer. Testing of levels of the biomarkers herein in combination with an evaluation of any additional biomedical information, including other laboratory tests (e.g., CA 19-9 testing, serum bilirubin concentration, CEA testing, alkaline phosphatase activity, presence of anemia), may, for example, improve sensitivity, or specificity for detecting pancreaticobiliary cancer (or other pancreatic cancer-related uses) as compared to biomarker testing alone or evaluating any particular item of additional biomedical information alone (e.g., ultrasound imaging alone). Additional biomedical information can be obtained from an individual using routine techniques known in the art, such as from the individual themselves by use of a routine patient questionnaire or health history questionnaire, etc., or from a medical practitioner, etc.

### Course of Treatment

In some embodiments, the biomarkers and methods herein find use in selecting a treatment course of action. For example, if the methods or biomarker levels herein indicate that a subject likely suffers from a pancreatobiliary cancer, a treatment course of action appropriate for that pancreatobiliary cancer is selected. If the methods or biomarker levels herein indicate that a subject likely suffers from a non-cancerous pancreatobiliary condition, a treatment course of action appropriate for that condition is selected. In some embodiments, the biomarkers and methods herein find use in monitoring a treatment for a pancreatobiliary cancer or other condition. Changes in the levels of the biomarkers during or after treatment may indicate relative success of failure of the treatment.

In one embodiment, methods are provided herein for treating a subject with pancreatobiliary cancer, monitoring treatment of pancreatobiliary cancer, and/or selecting a treatment for pancreatobiliary cancer. For example, in some embodiments the biomarkers described herein are used to select a subject for treatment with Gemcitabine or Cisplatin, or to monitor treatment therewith. Various cancer treatments that may find use in embodiments herein include surgery, radiation therapy, immunotherapy, endocrine therapy, gene therapy and administration of an anti-cancer agent.

Radiation therapy is the use of radiation to kill, destroy or treat the cancers. Exemplary radiation therapy includes, but is not limited to, gamma-radiation, neutron beam radiotherapy, electron beam radiotherapy, proton therapy, brachytherapy, and radioisotope therapy (i.e., systemic radioactive isotopes therapy).

An endocrine therapy is a treatment that adds, blocks or removes hormones. In one embodiment, the endocrine therapy comprises administration of natural hormones, synthetic hormones or other synthetic molecules that may block or increase the production of the body's natural hormones. In another embodiment, the endocrine therapy includes removal of a gland that makes a certain hormone.

As use herein, a gene therapy is the insertion of genes into a subject's cell and biological tissues to treat diseases, such as cancer. Exemplary gene therapy includes, but is not limited to, a germ line gene therapy, a somatic gene therapy, a CRISPR-based therapy, etc.

Immunotherapy (also called biological response modifier therapy, biologic therapy, biotherapy, immune therapy, or biological therapy) is treatment that uses parts of the immune system to fight disease. Immunotherapy can help the immune system recognize cancer cells, or enhance a response against cancer cells. Immunotherapies include active and passive immunotherapies. Active immunotherapies stimulate the body's own immune system while passive immunotherapies generally use immune system components created outside of the body. Examples of active immunotherapies include, but are not limited to vaccines including cancer vaccines, tumor cell vaccines (autologous or allogeneic), dendritic cell vaccines, antigen vaccines, anti-idiotype vaccines, DNA vaccines, viral vaccines, or Tumor-Infiltrating Lymphocyte (TIL) Vaccine with Interleukin-2 (IL-2) or Lymphokine-Activated Killer (LAK) Cell Therapy.

Examples of passive immunotherapies include but are not limited to monoclonal antibodies and targeted therapies containing toxins. Monoclonal antibodies include naked antibodies and conjugated monoclonal antibodies (also called tagged, labeled, or loaded antibodies). Naked monoclonal antibodies do not have a drug or radioactive material attached whereas conjugated monoclonal antibodies are joined to, for example, a chemotherapy drug (chemolabeled), a radioactive particle (radiolabeled), or a toxin (immunotoxin).

In some embodiments, an anti-cancer therapy includes administration of an anti-cancer agent. An “anti-cancer agent” is a compound, which when administered in an effective amount to a subject with cancer, can achieve, partially or substantially, one or more of the following: arresting the growth, reducing the extent of a cancer (e.g., reducing size of a tumor), inhibiting the growth rate of a cancer, and ameliorating or improving a clinical symptom or indicator associated with a cancer (such as tissue or serum components) or increasing longevity of the subject. Exemplary anti-cancer agents suitable for use in or with the methods described herein include any anti-cancer agents that have been approved for the treatment of cancer. In one embodiment, the anti-cancer agent includes, but is not limited to, a targeted antibody, an angiogenisis inhibitor, an alkylating agent, an antimetabolite, a vinca alkaloid, a taxane, a podophyllotoxin, a topoisomerase inhibitor, a hormonal antineoplastic agent and other antineoplastic agents. In one embodiment, the anti-cancer agents that can be used in methods described herein include, but are not limited to, paclitaxel, docetaxel, 5-fluorouracil, trastuzumab, lapatinib, bevacizumab, letrozole, goserelin, tamoxifen, cetuximab, panitumumab, gemcitabine, capecitabine, irinotecan, oxaliplatin, carboplatin, cisplatin, doxorubicin, epirubicin, cyclophosphamide, methotrexate, vinblastine, vincristine, melphalan, cytarabine, etoposide, daunorubicin, bleomycin, mitomycin and adriamycin and a combination thereof.

In some embodiments, the biomarkers and methods herein indicate that a subject does not suffer from pancreatobiliary cancer, but instead suffers from another pancreatobiliary condition. In such embodiment, methods are provided herein for treating a subject with a non-cancerous pancreatobiliary condition, monitoring treatment of a non-cancerous pancreatobiliary condition, and/or selecting a treatment for a non-cancerous pancreatobiliary condition. In some embodiments, a non-cancerous pancreatobiliary condition is a gallbladder disease or condition such as cholelithiasis, cholecystitis, an infection of the gallbladder, an obstruction of the gallbladder, etc. In some embodiments, a non-cancerous pancreatobiliary condition is a pancreatic disease or condition such as Pancreatitis (acute and chronic inflammation of the pancreas), cystic pancreatic tumor, pseudocysts, stricture, etc. In some embodiments, a non-cancerous pancreatobiliary condition is a pancreatic disease or condition such as choledocholithiasis, strictures, cysts, cholangitis, etc. In some embodiments, the biomarkers and methods herein provide for diagnosing and/or prognosing one or more of the above non-cancerous conditions. In some embodiments, the biomarkers and methods herein provide for selecting a treatment course of action for one or more of the above non-cancerous conditions. In some embodiments, the biomarkers and methods herein provide for monitoring treatment of one or more of the above non-cancerous conditions.

Embodiments herein find use in various clinical applications, such as point of care diagnosis of malignant and benign biliary strictures, non-invasive diagnosis for malignant and benign biliary strictures, monitoring chemotherapy treatment for pancreaticobiliary and hepatobiliary cancers, chemotherapy management for pancreaticobiliary and hepatobiliary cancers, cancer surveillance for patients with primary sclerosing cholangitis, cancer surveillance for patients with cirrhosis, graft function monitoring in patients with liver transplant, cancer surveillance for patients that had liver transplanted for hepatocellular carcinoma, etc.

Embodiments herein provide a variety of significant advantages over existing diagnostic and prognostic techniques, such as: being less invasive than current methods of diagnosis which requires biopsy using needles or brushings which can cause trauma in the liver, bile ducts, and pancreas resulting in severe complications; increased sensitivity in diagnosing cancer thus reducing repeat biopsies and hospital visits; early detection of cancer or precancerous lesions in patients with primary sclerosing cholangitis, which is currently non-existent.

### EXPERIMENTAL

### Example 1

### Metabolomics

Experiments were conducted during development of embodiments herein to demonstrate detectable distinctions in the metabolome of subjects with pancreaticobiliary malignancies vs. benign biliary disease.

19 patients were identified that fulfilled our study inclusion criteria. Of these 19 patients, ERCP indications include PSC (Ref. 1; incorporated by reference in its entirety), sphincter of oddi dysfunction (SOD) (Ref. 4; incorporated by reference in its entirety), new pancreaticobiliary malignancy (Ref. 5; incorporated by reference in its entirety), choledocholithiasis (Ref. 8; incorporated by reference in its entirety), and external compression (Ref. 1;

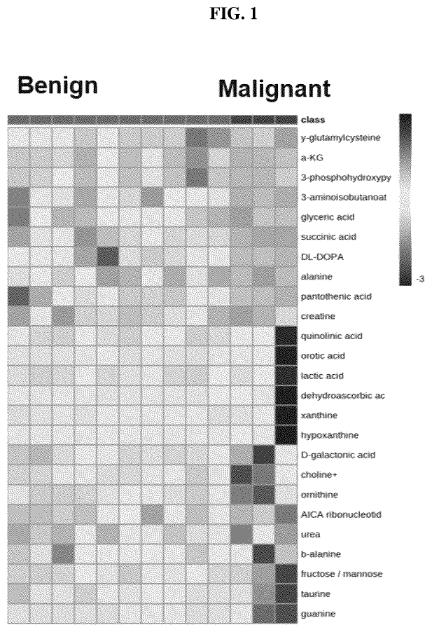

incorporated by reference in its entirety) (FIG. 1). Metabolites were extracted from bile samples from patients with new pancreaticobiliary malignancy and benign biliary disease (e.g., SOD, choledocholithiasis) and submitted them for comprehensive hydrophilic metabolomics utilizing LCMS.

For metabolomics of bile 0.1 mL of bile was homogenized in 1 mL of ice-cold 80% methanol with a Tissue Lyser II (Qiagen) (3 sets of 2 minutes on maximum speed, with 2 minutes of rest on dry ice in between sets). The homogenized tissue was then incubated at −80° C. for 15 minutes and then centrifuged at 18,000×g at 4° C. for 5 minutes. The lipid layer was avoided, and the supernatant was transferred to a new tube on dry ice and then centrifuged at 18,000×g at 4 C for 2 minutes. The equivalent volume of 20 mg of tissue was transferred to a new tube, which was then dried in a speed-vac (Thermo Fisher).

### LCMS Analysis of Metabolites

The dried metabolites were reconstituted in 60% acetonitrile followed by overtaxing for 30 seconds and then centrifugation for 30 min at 20,000 xg at 4° C. Supernatant was analyzed by High-Performance Liquid Chromatography and High-Resolution Mass Spectrometry and Tandem Mass Spectrometry (HPLC-MS/MS). Specifically, system consisted of a Thermo Q-Exactive in line with an electrospray source and an Ultimate3000 (Thermo) series HPLC consisting of a binary pump, degasser, and auto-sampler outfitted with a Xbridge Amide column (Waters; dimensions of 3.0 mm×100 mm and a 3.5 μm particle size). The mobile phase A contained 95% (vol/vol) water, 5% (vol/vol) acetonitrile, 10 mM ammonium hydroxide, 10 mM ammonium acetate, pH=9.0; B was 100% Acetonitrile. The gradient was as following: 0 min, 15% A; 2.5 min, 30% A; 7 min, 43% A; 16 min, 62% A; 16.1-18 min, 75% A; 18-25 min, 15% A with a flow rate of 150 L/min. The capillary of the ESI source was set to 275° C., with sheath gas at 35 arbitrary units, auxiliary gas at 5 arbitrary units and the spray voltage at 4.0 kV. In positive/negative polarity switching mode, an m/z scan range from 60 to 900 was chosen and MS1 data was collected at a resolution of 70,000. The automatic gain control (AGC) target was set at 1×10⁶ and the maximum injection time was 200 ms. The top 5 precursor ions were subsequently fragmented, in a data-dependent manner, using the higher energy collisional dissociation (HCD) cell set to 30% normalized collision energy in MS2 at a resolution power of 17,500. Besides matching m/z, metabolites are identified by matching either retention time with analytical standards and/or MS2 fragmentation pattern. Data acquisition and analysis were carried out by Xcalibur 4.1 software and Tracefinder 4.1 software, respectively (both from Thermo Fisher Scientific). Metabolite concentrations within each sample were normalized to total ion count. For stable isotope metabolomics, data was corrected for natural abundance and tracer purity using IsoCorrectoR (29).

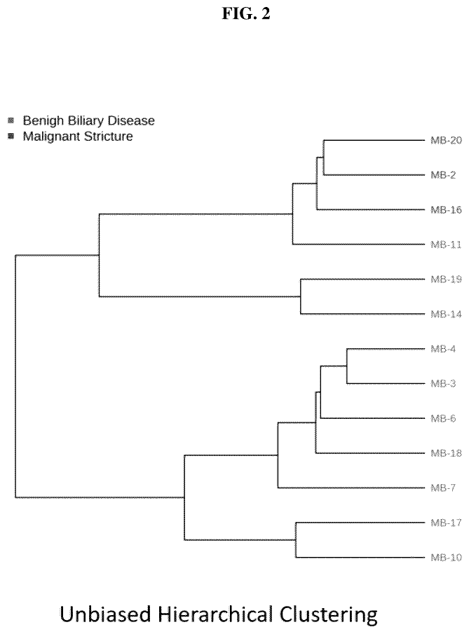

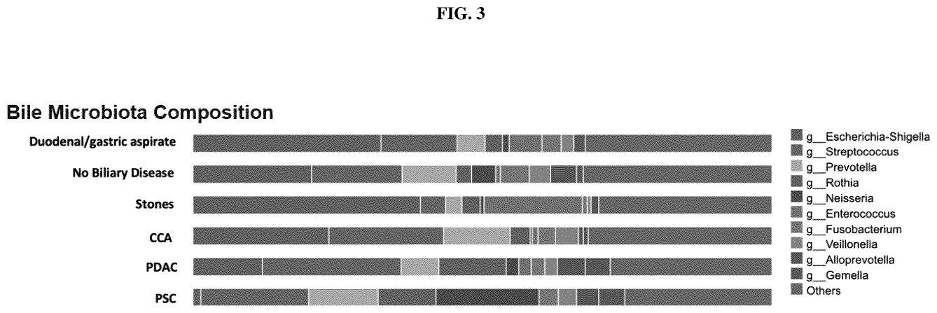

The biliary metabolomic profile of the three patients with new pancreaticobiliary malignancy (2 pancreatic cancer and 1 CCA) were compared with the 10 patients with benign biliary disease as non-malignant controls, to assess whether metabolite levels differ between the two groups. Analysis utilizing the MetaboAnalyst package in R software (Ref. 19; incorporated by reference in its entirety) demonstrated 37 statistically significant (p<0.05) differentially abundant metabolites between the groups (FIG. 1A-B). Of the 37 significant changes in metabolites, 30 were significantly increased in malignancy samples and 7 were significantly decreased (FIG. 1A-B). Principle-component analysis (PCA) of bile metabolomics revealed a clear separation between patients with new pancreaticobiliary malignancy compared to patients with benign biliary disease, demonstrating that bile metabolomic profiles are distinct in pancreaticobiliary malignancy (FIG. 2A). Hierarchical clustering analysis of the bile metabolomics data showed discrete grouping of metabolites from patients with malignancy and those with benign biliary disease (FIG. 2B). Within the malignancy group, the biliary metabolic profiles of the 2 samples of pancreatic cancer were more strongly associated when compared to that of intra-ductal cholangiocarcinoma (FIG. 2B).

Experiments conducted during development of embodiments herein analyzing bile metabolomics identified clear differences in the abundance of biliary metabolites in patients with malignancy compared to those with benign biliary disease. This reveals the bile metabolome is distinct in pancreaticobiliary malignancy. In addition, the biliary metabolomic profile is sensitive enough to differentiate patients with malignant stricture from new pancreatic cancer and intra-ductal cholangiocarcinoma.

Experiments were conducted during development of embodiments herein to demonstrate detectable distinctions in the microbiome of subjects with pancreaticobiliary malignancies vs. benign biliary disease.

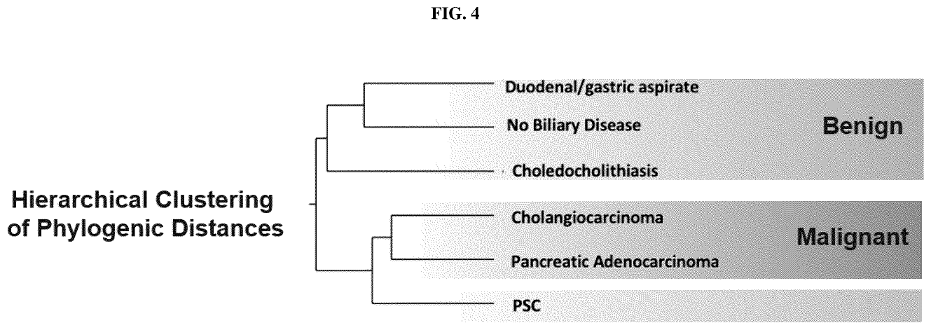

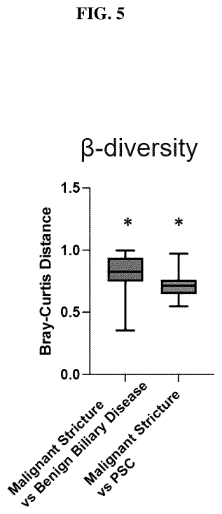

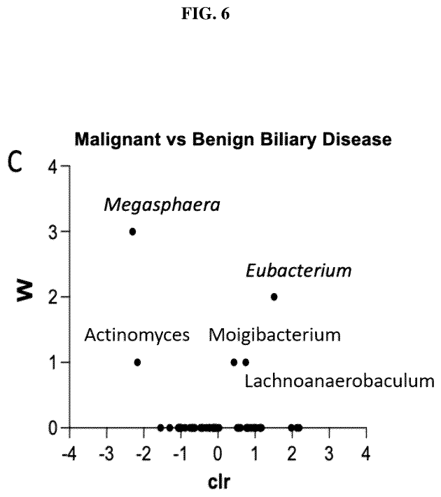

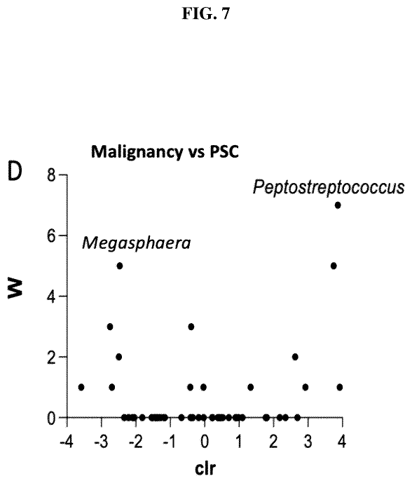

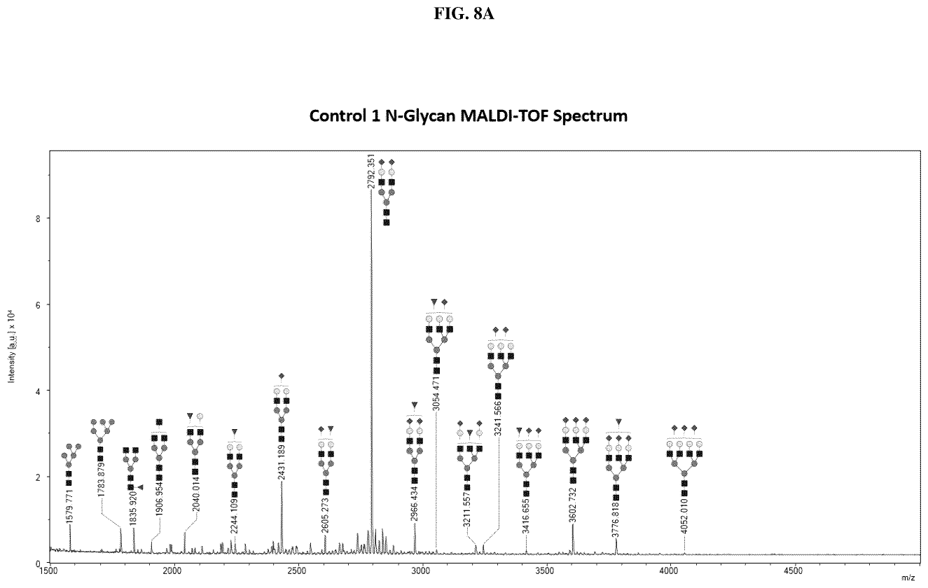

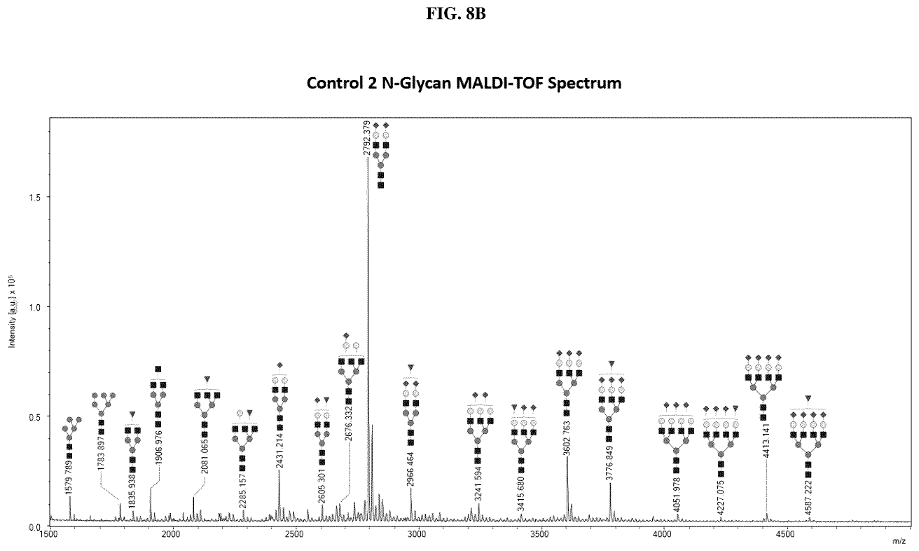

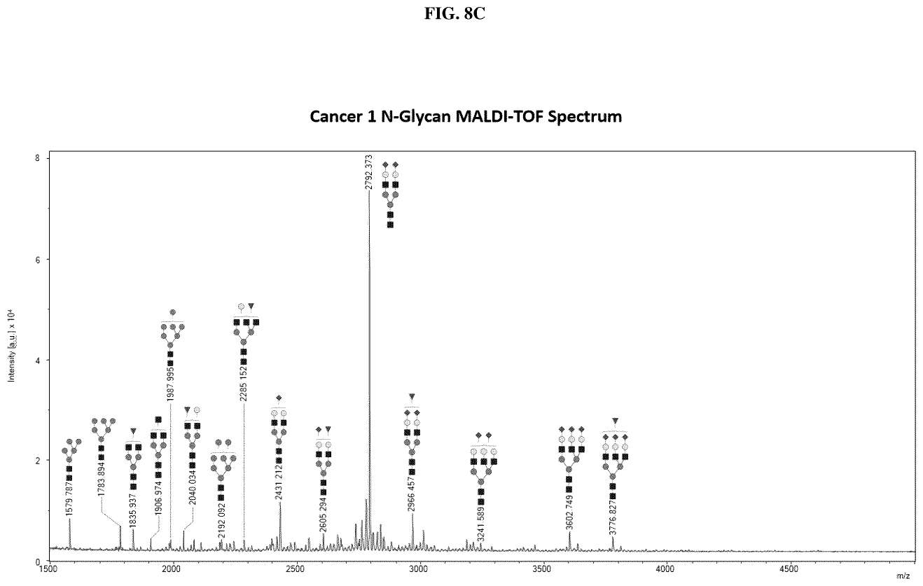

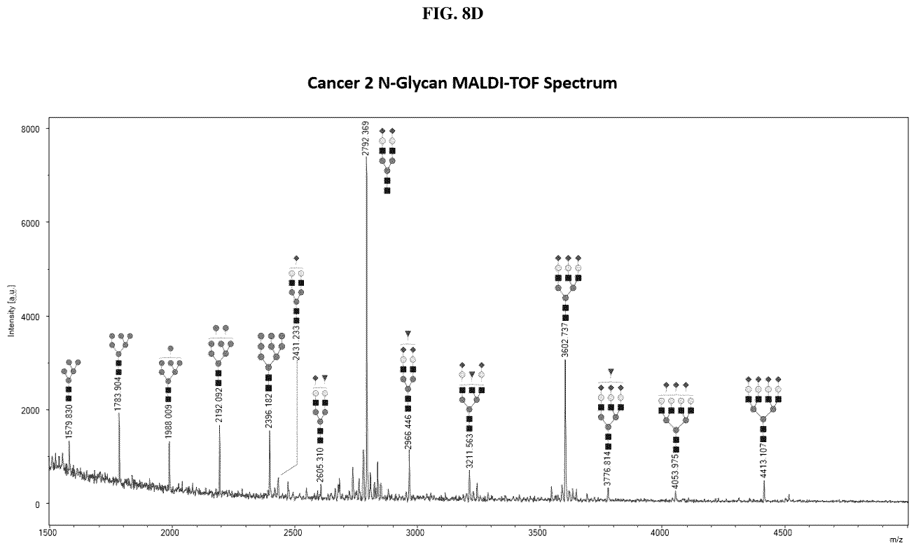

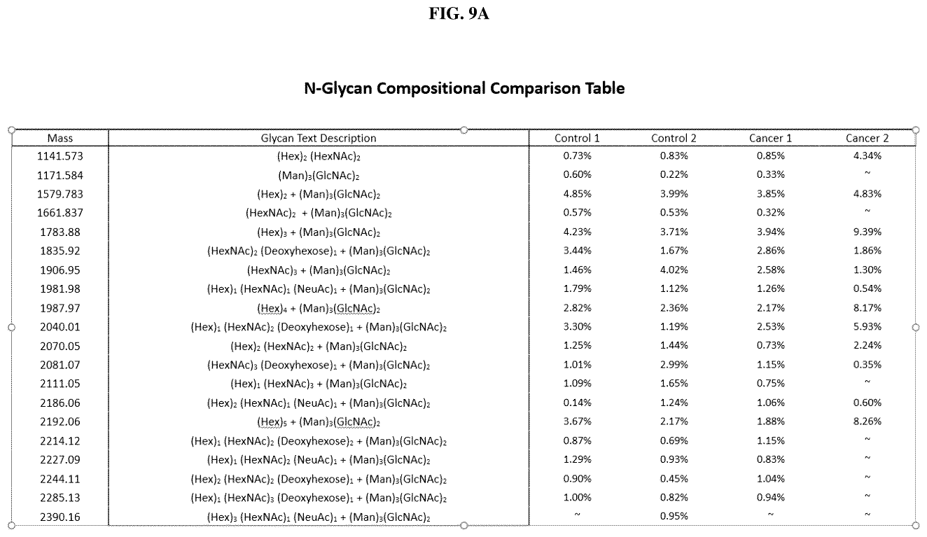

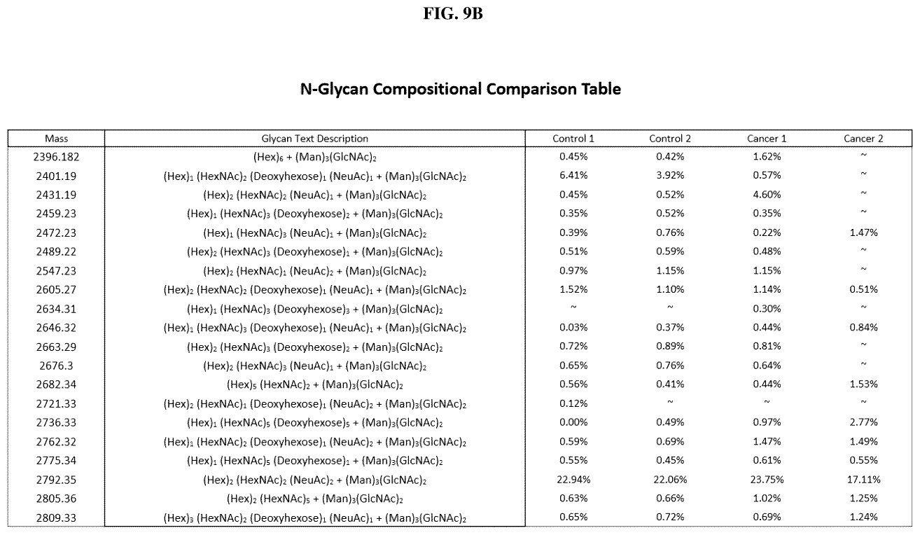

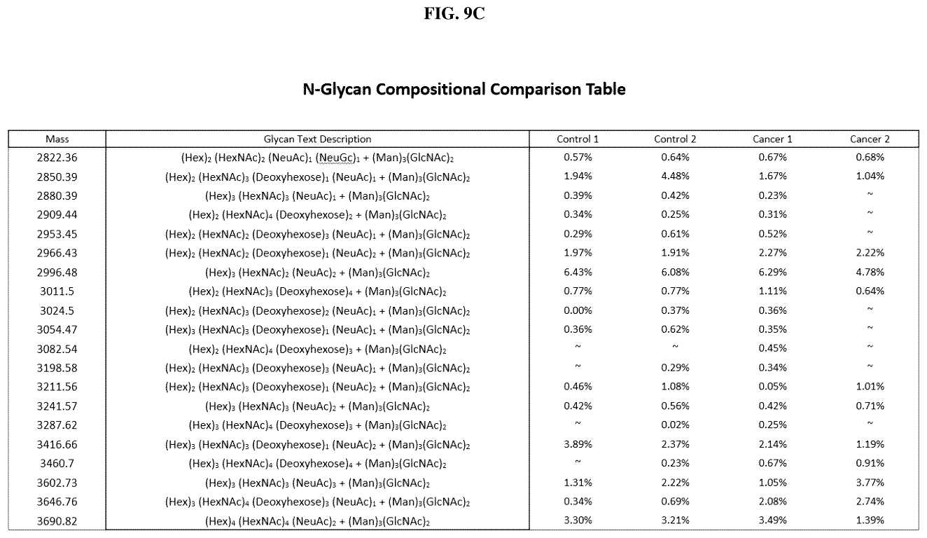

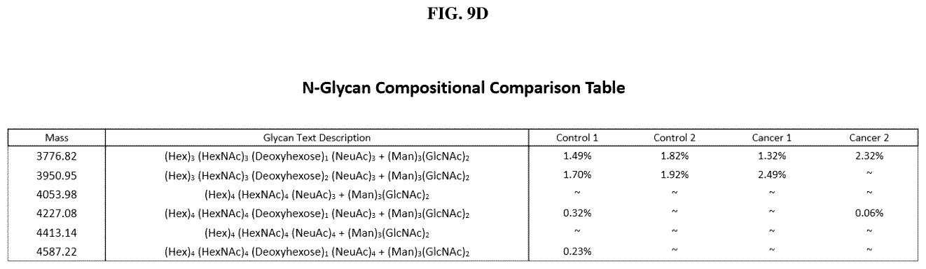

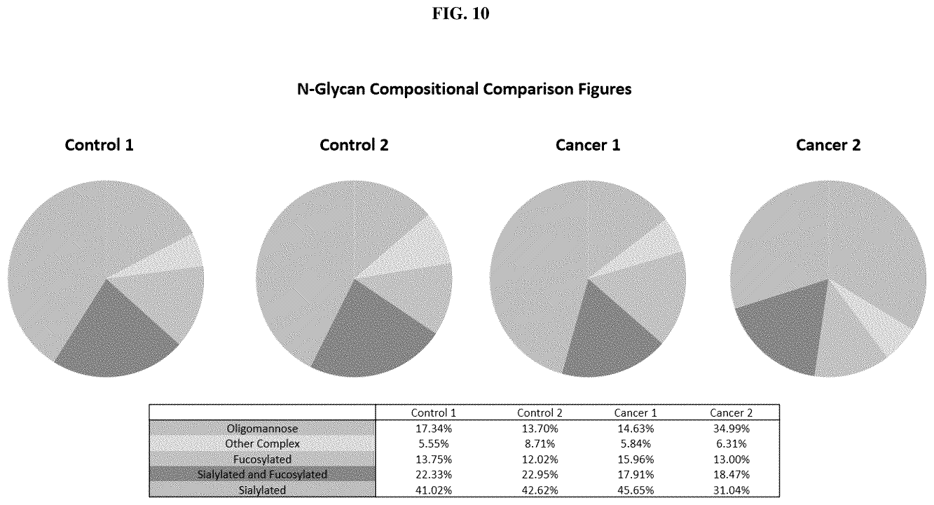

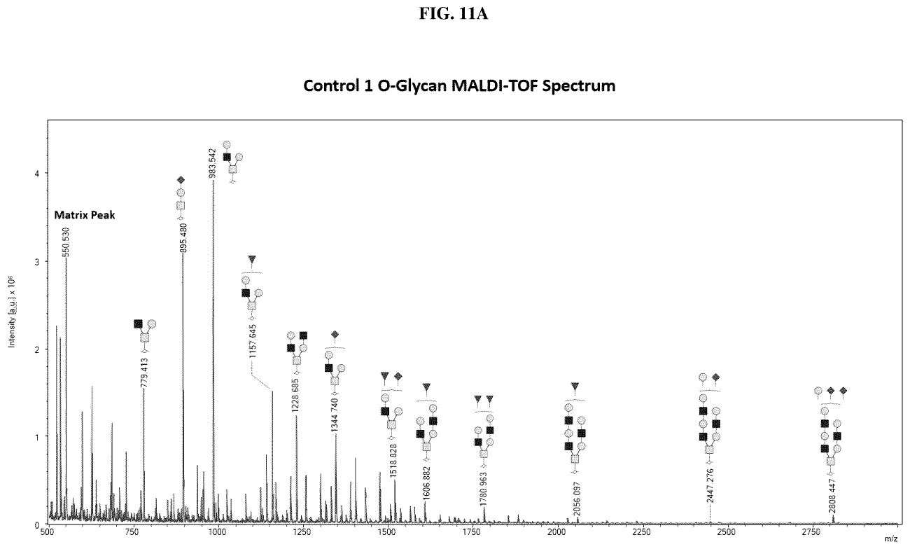

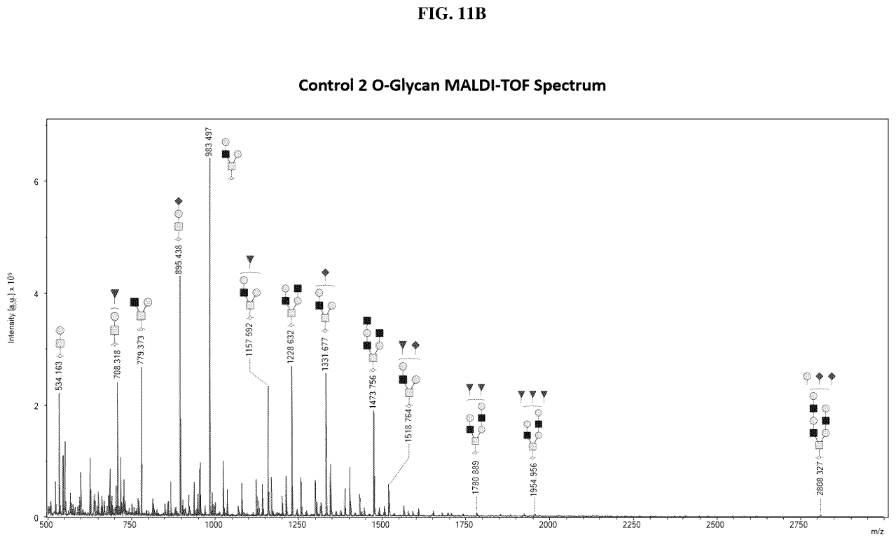

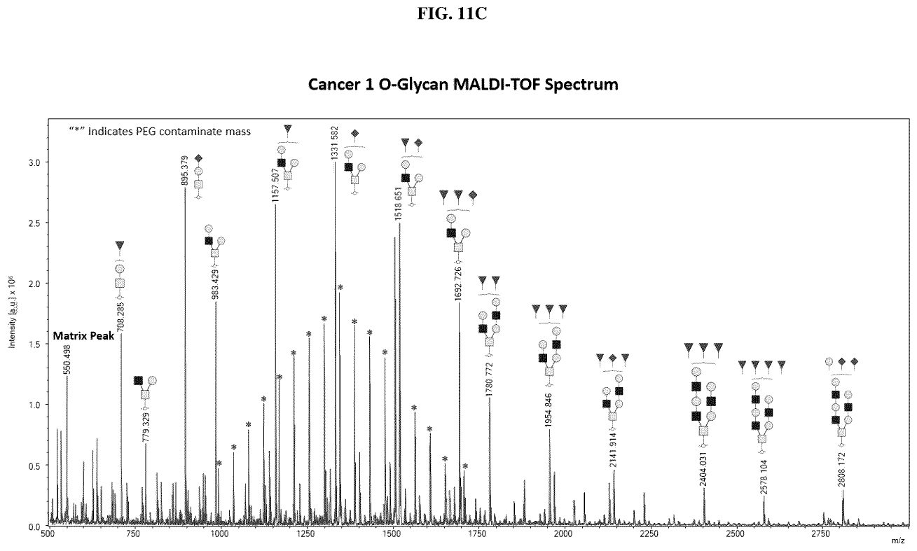

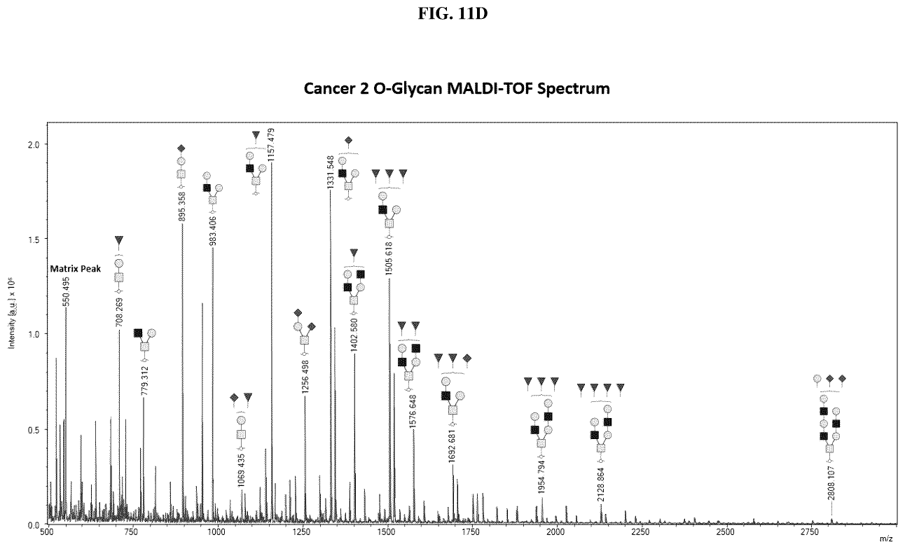

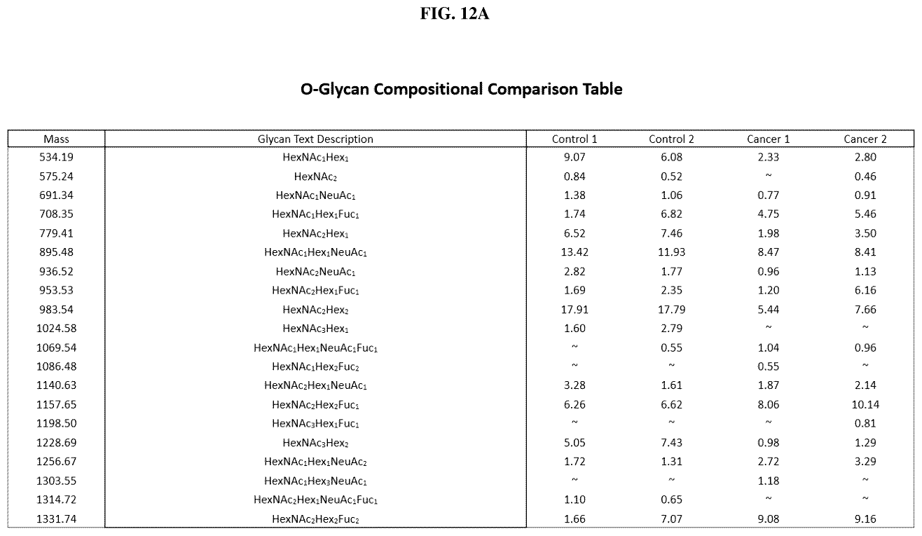

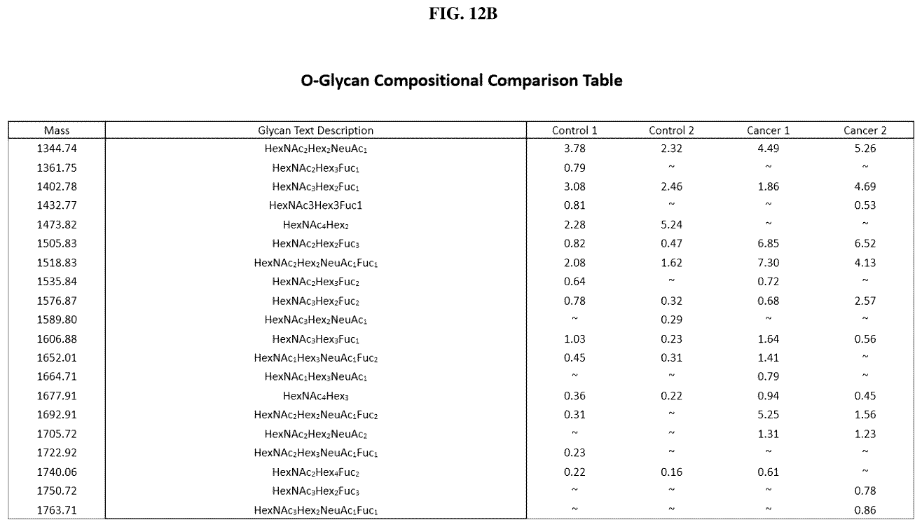

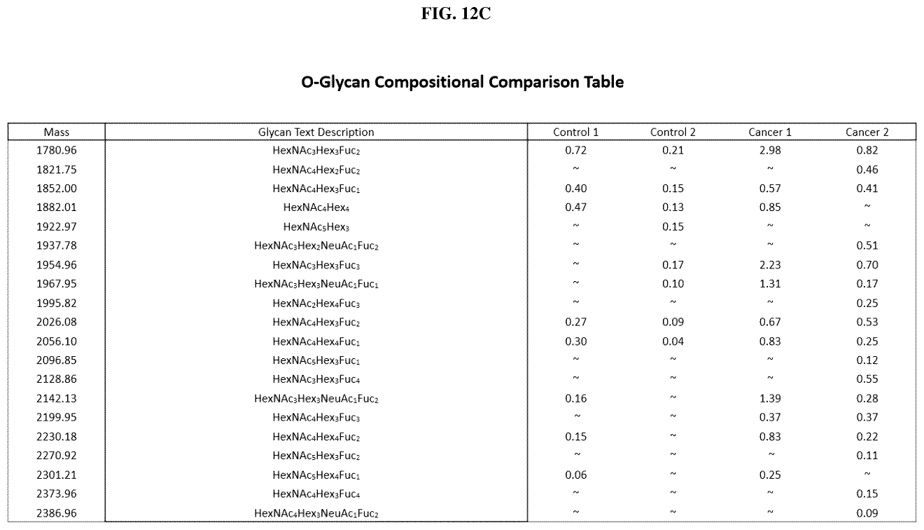

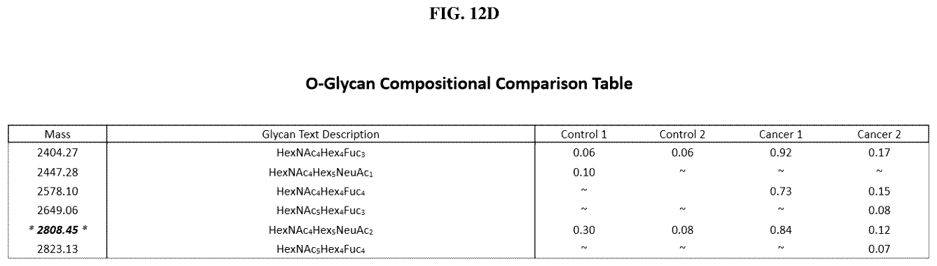

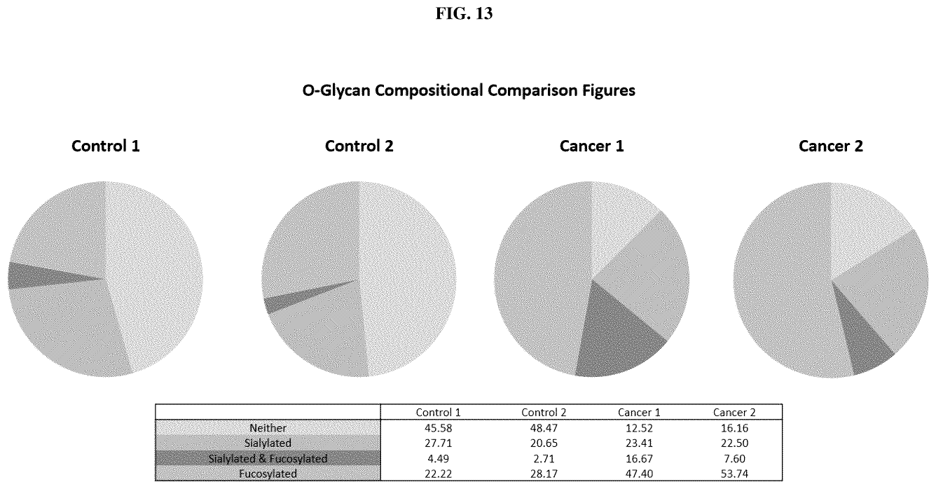

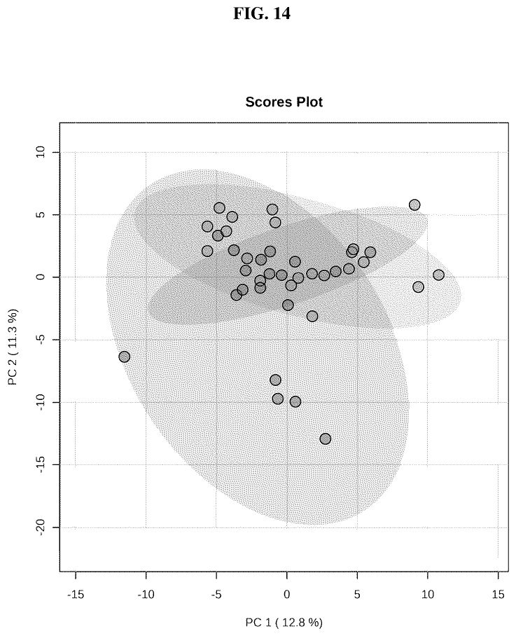

Bile was obtained from patients with native papilla undergoing ERCP for indication of biliary obstruction with suspicion of new pancreatic cancer or cholangiocarcinoma (n=11), benign biliary diseases (i.e. choledocholithiasis, sphincter of oddi dysfunction) (n=17) and primary sclerosing cholangitis (PSC) (n=4). Bacterial DNA was extracted from bile samples using AMPure XP beads and 16S rRNA sequencing was performed on PCR products of V3-V4 hypervariable regions. PCR products were sequenced and data analysis and visualization were performed on QIIME2 pipeline (FIG. 3-7).

### Example 2

### Glyomics

### Materials & Methods

### Delipidation of Samples:

3 mL of 2:1 MeOH:CHCl₃ were added to each sample and the samples were shaken for 2-3 minutes. Next, 1 mL of CHCl₃ and 1.8 mL of water were added to each sample before being shaken for another 2-3 minutes followed by centrifugation for −5 minutes to promote phase separation. The upper aqueous phase was removed while being careful to not disturb either the interphase or organic phase. To each sample, an equivalent volume of MeOH was added to each sample before being shaken for 2-3 minutes before centrifuging again for −10 minutes to pellet precipitated proteins. The organic phase was removed from the samples and keep, and the remaining protein pellet was dried under N₂. The protein fractions were then resuspended in NH₄HCO₃ before proceeding to the release of N-glycans.

### Enzymatic Release of N-Glycans:

Delipidated samples were transferred to a clean Eppendorf tube where an equivalent volume of 25 mM DTT was added and incubated at 50° C. for 45 minutes to denature. The samples were then gradually desalted using 10 kDa cutoff spin filters by centrifuging at 14,000×g for 15 minutes. The samples were then washed using 400 μL of 50 mM ammonium bicarbonate and centrifuged again at 14,000×g for 15 minutes. This step was repeated once more. The sample remaining in the filter was transferred to a clean Eppendorf tube and the N-glycans were released from proteins by digestion with PNGase F and incubating at 37° C. for 18 hours. The released N-glycans were separated from the O-glycoprotein using 10 kDa cutoff spin filter, purified using C18 cartridge, and were lyophilized before proceeding to permethylation (N-glycans) or P-elimination (0-glycoproteins).

### β-Elimination of O-Glycans:

Lyophilized O-glycoproteins that did not flow through 10 kDa spin filter were resuspended in 50 mM NaOH. pH was checked to confirm basic conditions. 19 mg/250 uL NaBH₄ in 50 mM NaOH was added, solution was incubated at 50° C. for 18 h. Samples were then cooled to RT and neutralized by adding 10% acetic acid dropwise. Samples were then passed through DOWEX H⁺ resin column and C18 column. Samples were then lyophilized, and borates were removed using a solution of 9:1 methanol to acetic acid under a stream of N₂.

### Per-O-Methylation of Glycans:

Released glycans were permethylated by using methyl iodide on DMSO/NaOH mixture. Briefly, the dried eluate was dissolved with dimethyl sulfoxide and methylated by using methyl iodide on DMSO/NaOH mixture. The reaction was quenched with water and the reaction mixture was extracted with dichloromethane and dried. The dried glycans were re-dissolved in methanol and profiled by MALDI-TOF.

MALDI-TOF analysis of Per-O-methylated Glycans:

2 μL of permethylated N-glycans were transferred to a clean Eppendorf tube and mixed with an equivalent volume of a saturated solution of DHB (15 mg/mL DHB, 70% acetonitrile, 30% water, 0.1% formic acid) and were spotted onto MALDI target plate. Samples were then run on a Bruker rapifleX Tissuetyper in reflector positive ion mode.

### Results

All masses provided in the figures and the tables for both the N and O glycans are sodium adducts [M+Na].

In the N-glycan fractions, the control bile and the cancer bile samples exhibited similar relative compositions of sialylated glycan structures, with sialylated structures comprising 41-46% of the total glycan relative abundance in samples Control 1, Control 2, and Cancer 1, while comprising only 31.04% in Cancer 2. Additionally, the relative composition of oligomannose structures was significantly greater in Cancer 2 at 35% whereas all other samples exhibited a relative abundance of 13-18%. The number of individual N-glycan masses observed in each sample was n=58 for Control 1, n=59 for Control 2, n=60 for Cancer 1, and n=36 for Cancer 2.

In the O-glycan fraction, there was a distinct increase in the relative composition of fucosylated glycan structures in the cancer bile samples compared to the control bile samples. The control bile samples exhibited 22.22% (Control 1) and 28.17% (Control 2) fucosylation while the cancer bile samples exhibited 47.40% (Cancer 1) and 53.74% (Cancer 2). Additionally, there was a notable increase in the relative composition of structures that are both fucosylated and sialylated in the cancer bile samples, with the control bile samples exhibiting 4.49% (Control 1) and 2.71% (Control 2) while the cancer bile samples exhibiting 16.67% (Cancer 1) and 7.60% (Cancer 2). The relative composition of structures that were neither sialylated nor fucosylated decreased in the cancer bile samples when compared to the control bile samples, while the relative composition of only sialylated structures remained comparable across all 4 samples. The number of individual O-glycan masses observed in each sample was n=42 for Control 1, n=38 for Control 2, n=43 for Cancer 1, and n=50 for Cancer 2. O-glycan masses observed in both control bile samples but neither cancer bile samples are masses 1024 and 1314. Masses observed in both cancer bile samples but neither control bile samples are masses 1705, 2200, and 2578.

### Example 3

### Exemplary Metabolite Signature of Cholangiocarcinoma

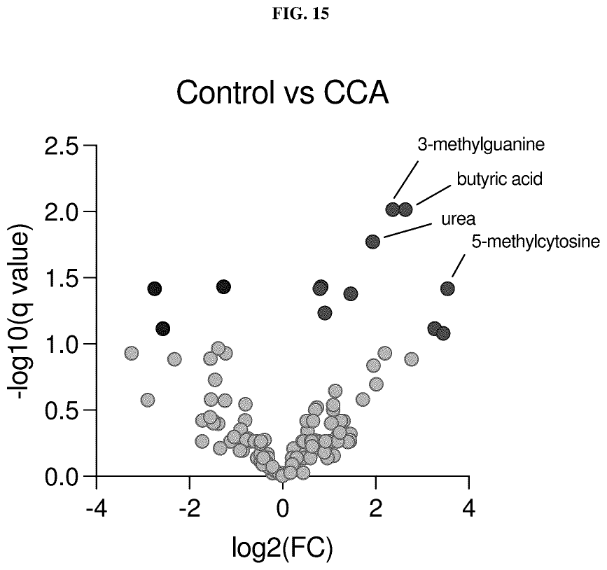

Polar metabolomics were performed of bile collected from patients with cholangiocarcinoma (bile duct cancer), gallstone disease, and no biliary disease and unique metabolites that are upregulated in cholangiocarcinoma were identified (Table 1; FIG. 15).

TABLE 1
Exemplary metabolites verified as significantly
upregulated (p < 0.05) in cholangiocarcinoma.

| metabolite name | fold over benign | p-value |
|---|---|---|
| 3-/7-methylguanine | 7.95210518 | 0.00418001 |
| sucrose | 2.023893875 | 0.02601831 |
| 5-methylcytosine | 15.0566372 | 0.00399068 |
| tryptamine | 8.075962535 | 0.00268317 |
| N-methyltryptamine | 6.447571726 | 0.00621176 |
| 1-phenylethylamine | 1.437030267 | 0.02081676 |
| pyruvic acid | 4.03046359 | 0.06522307 |
| deoxyadenosine | 39.77126551 | 0.00099502 |
| dCTP | 31.56847549 | 0.00032316 |
| picolinic acid | 1.895111268 | 3.2645E−05 |
| guanidinoacetate | 2.110315531 | 0.05790081 |
| NADPH | 9.454072546 | 0.02131271 |
| 5-methylthioadenosine | 2.205828689 | 0.02908797 |
| deoxyuridine | 16.67894637 | 0.03379107 |
| D-sedoheptulose-7-phosphate | 31.66999287 | 0.00076568 |
| aspartic acid | 2.873544522 | 0.014442 |
| lactose/maltose | 3.222281622 | 0.00112561 |
| uric acid | 0.606407567 | 0.015383 |
| phenylalanine | 2.351820824 | 0.04558191 |
| glutamate | 2.509712292 | 0.00497708 |
| UMP | 70.03937113 | 0.0047433 |
| uracil | 5.456147306 | 0.00167723 |
| urea | 3.166770932 | 4.1927E−05 |
| uric acid | 0.606407567 | 0.015383 |
| uridine | 4.086590835 | 0.0003904 |
| xanthine | 5.11773411 | 0.00163577 |

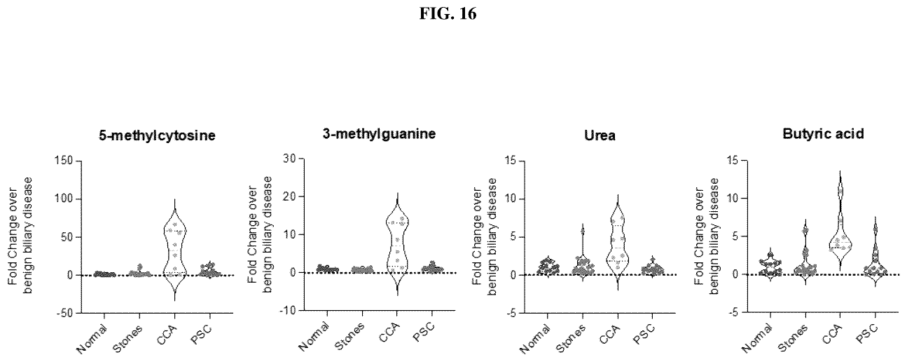

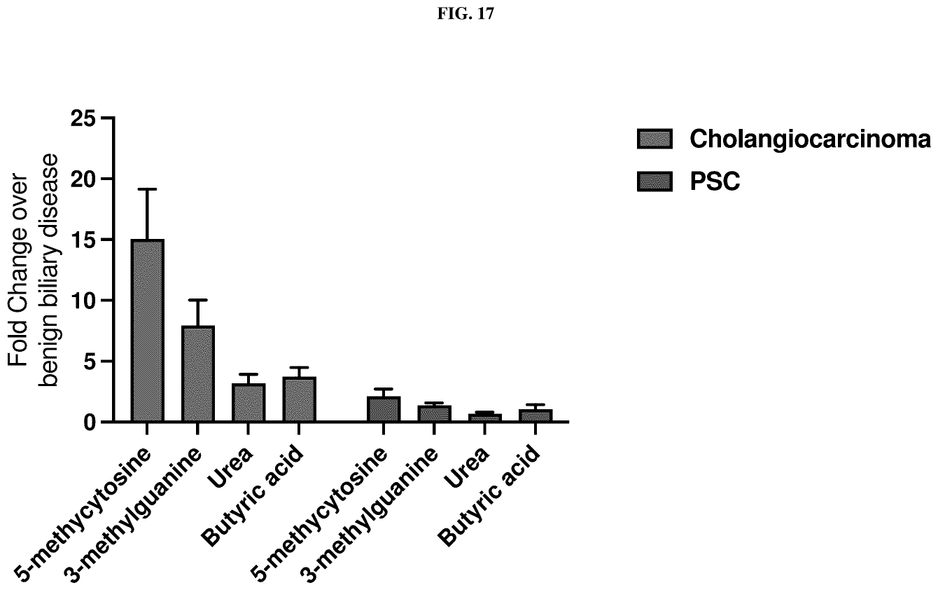

Two of the metabolites are methylated nucleotides (5-methylcytosine, 3-methylguanine) from cell-free DNA obtained from bile and appear to be specific to cholangiocarcinoma (FIGS. 15-17). Urea is upregulated in hepatobiliary cancers. Cancer cell metabolism shifts nutrient use to pyruvate/glycolysis and decreases oxidation of short-chain fatty acids such as butyrate, leading to butyrate elevation.

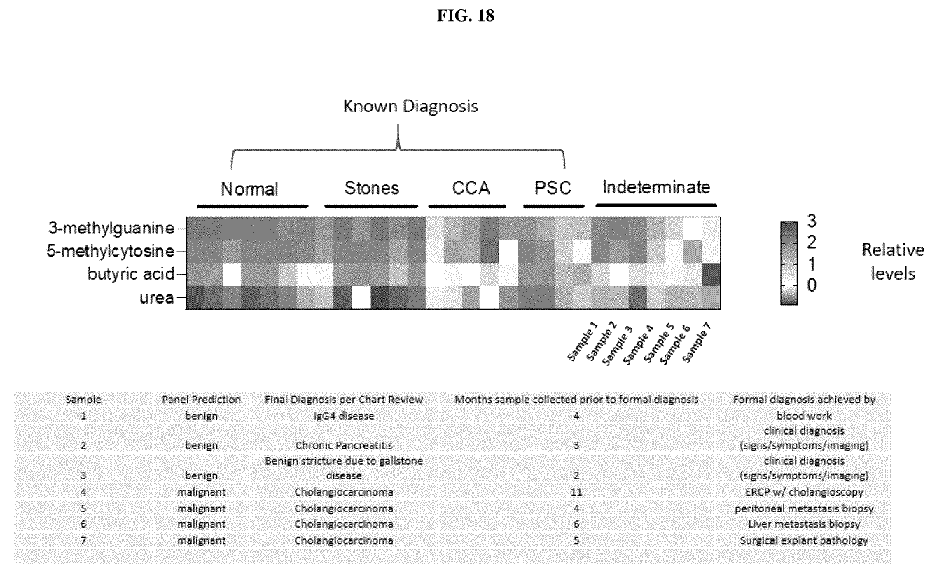

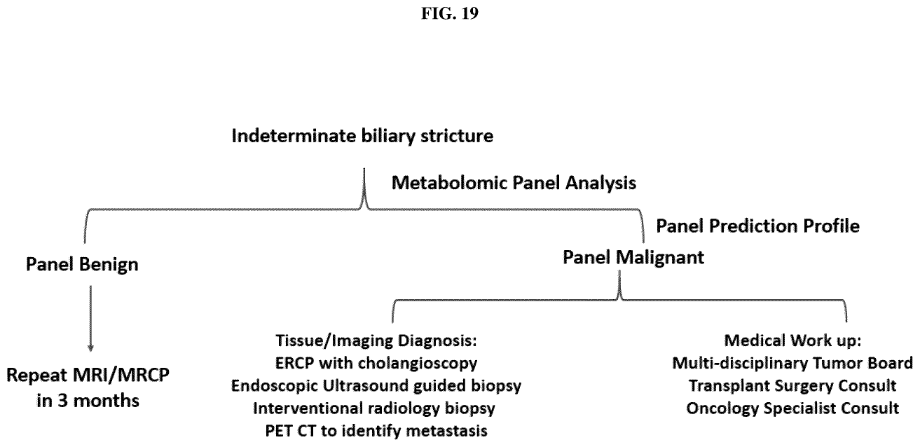

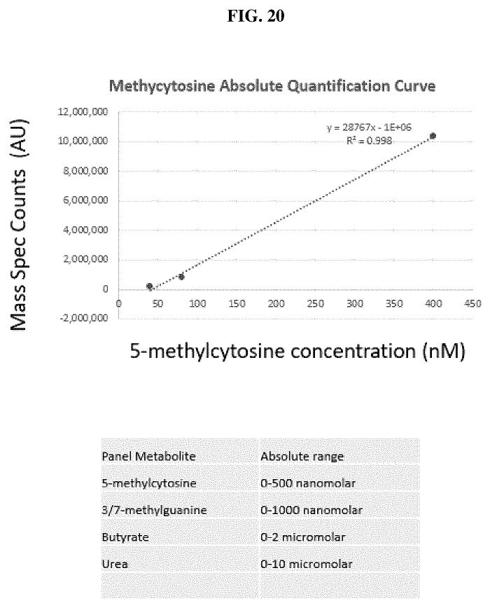

A diagnostic panel was created using these 4 metabolites for evaluating bile from patients with indeterminate biliary strictures. Indeterminate biliary strictures are defined as narrowing in the bile duct that are inconclusive on pathology/cytology results, they are diagnostic challenges for gastroenterologists and oncologists. Current tests have low diagnostic sensitivity and prevent early diagnosis and usually delay treatment for malignant strictures resulting in increasing health-care costs and poor patient outcomes. Using the exemplary 4-metabolite panel, the categorization of 10 indeterminate strictures (5 benign and 5 malignant) we accurately predicted. The findings were confirmed by autopsy/surgical pathology results from medical records of these patients (FIG. 18).

These findings demonstrate that panels of biomarkers can be made from the biomarkers described herein and used to discriminate benign from malignat strictures. These finding further highlight the importance of methylation patterns of cell-free DNA in bile as a strong predictor of hepatobiliary malignancy.

### REFERENCES

The following references, some of which are cited above by number, are herein incorporated by reference in their entireties.
1. Mavros M N, Economopoulos K P, Alexiou V G, Pawlik T M. Treatment and Prognosis for Patients With Intrahepatic Cholangiocarcinoma: Systematic Review and Meta-analysis. JAMA Surg. 2014; 149(6):565-74. Epub 2014/04/11. doi: 10.1001/jamasurg.2013.5137. PubMed PMID: 24718873.
2. Lee Y M, Kaplan M M. Primary sclerosing cholangitis. N Engl J Med. 1995; 332(14):924-33. Epub 1995/04/06. doi: 10.1056/NEJM199504063321406. PubMed PMID: 7877651.
3. Chalasani N, Baluyut A, Ismail A, Zaman A, Sood G, Ghalib R, McCashland T M, Reddy K R, Zervos X, Anbari M A, Hoen H. Cholangiocarcinoma in patients with primary sclerosing cholangitis: a multicenter case-control study. Hepatology. 2000; 31(1):7-11. Epub 1999/12/29. doi: 10.1002/hep.510310103. PubMed PMID: 10613720.
4. Razumilava N, Gores G J, Lindor K D. Cancer surveillance in patients with primary sclerosing cholangitis. Hepatology. 2011; 54(5):1842-52. Epub 2011/07/28. doi: 10.1002/hep.24570. PubMed PMID: 21793028; PMCID: PMC3205332.
5. Dorrestein P C, Mazmanian S K, Knight R. Finding the missing links among metabolites, microbes, and the host. Immunity. 2014; 40(6):824-32. Epub 2014/06/21. doi: 10.1016/j.immuni.2014.05.015. PubMed PMID: 24950202; PMCID: PMC4503329.
6. Pittayanon R, Lau J T, Leontiadis G I, Tse F, Yuan Y, Surette M, Moayyedi P. Differences in Gut Microbiota in Patients With vs Without Inflammatory Bowel Diseases: A Systematic Review. Gastroenterology. 2020; 158(4):930-46 el. Epub 2019/12/10. doi: 10.1053/j.gastro.2019.11.294. PubMed PMID: 31812509.
7. Feng Q, Liang S, Jia H, Stadlmayr A, Tang L, Lan Z, Zhang D, Xia H, Xu X, Jie Z, Su L, Li X, Li X, Li J, Xiao L, Huber-Schonauer U, Niederseer D, Xu X, Al-Aama J Y, Yang H, Wang J, Kristiansen K, Arumugam M, Tilg H, Datz C, Wang J. Gut microbiome development along the colorectal adenoma-carcinoma sequence. Nat Commun. 2015; 6:6528. Epub 2015/03/12. doi: 10.1038/ncomms7528. PubMed PMID: 25758642.
8. Lopetuso L R, Severgnini M, Pecere S, Ponziani F R, Boskoski I, Larghi A, Quaranta G, Masucci L, Ianiro G, Camboni T, Gasbarrini A, Costamagna G, Consolandi C, Cammarota G. Esophageal microbiome signature in patients with Barrett's esophagus and esophageal adenocarcinoma. PLoS One. 2020; 15(5):e0231789. Epub 2020/05/06. doi: 10.1371/journal.pone.0231789. PubMed PMID: 32369505; PMCID: PMC7199943.
9. Shen H, Ye F, Xie L, Yang J, Li Z, Xu P, Meng F, Li L, Chen Y, Bo X, Ni M, Zhang X. Metagenomic sequencing of bile from gallstone patients to identify different microbial community patterns and novel biliary bacteria. Sci Rep. 2015; 5:17450. Epub 2015/12/03. doi: 10.1038/srep17450. PubMed PMID: 26625708; PMCID: PMC4667190.
10. Pavlova N N, Thompson C B. The Emerging Hallmarks of Cancer Metabolism. Cell Metab. 2016; 23(1):27-47. Epub 2016/01/16. doi: 10.1016/j.cmet.2015.12.006. PubMed PMID: 26771115; PMCID: PMC4715268.
11. Franzosa E A, Sirota-Madi A, Avila-Pacheco J, Fornelos N, Haiser H J, Reinker S, Vatanen T, Hall A B, Mallick H, McIver L J, Sauk J S, Wilson R G, Stevens B W, Scott J M, Pierce K, Deik A A, Bullock K, Imhann F, Porter J A, Zhernakova A, Fu J, Weersma R K, Wijmenga C, Clish C B, Vlamakis H, Huttenhower C, Xavier R J. Gut microbiome structure and metabolic activity in inflammatory bowel disease. Nat Microbiol. 2019; 4(2):293-305. Epub 2018/12/12. doi: 10.1038/s41564-018-0306-4. PubMed PMID: 30531976; PMCID: PMC6342642.
12. Patel N, Benipal B. Incidence of Cholangiocarcinoma in the USA from 2001 to 2015: A U S Cancer Statistics Analysis of 50 States. Cureus. 2019; 11(1):e3962. Epub 2019/04/09. doi: 10.7759/cureus.3962. PubMed PMID: 30956914; PMCID: PMC6436669.
13. Florio A A, Ferlay J, Znaor A, Ruggieri D, Alvarez C S, Laversanne M, Bray F, McGlynn K A, Petrick J L. Global trends in intrahepatic and extrahepatic cholangiocarcinoma incidence from 1993 to 2012. Cancer. 2020; 126(11):2666-78. Epub 2020/03/05. doi: 10.1002/cncr.32803. PubMed PMID: 32129902; PMCID: PMC7323858.
14. Banales J M, Marin J J G, Lamarca A, Rodrigues P M, Khan S A, Roberts L R, Cardinale V, Carpino G, Andersen J B, Braconi C, Calvisi D F, Perugorria M J, Fabris L, Boulter L, Macias R I R, Gaudio E, Alvaro D, Gradilone S A, Strazzabosco M, Marzioni M, Coulouarn C, Fouassier L, Raggi C, Invernizzi P, Mertens J C, Moncsek A, Rizvi S, Heimbach J, Koerkamp B G, Bruix J, Forner A, Bridgewater J, Valle J W, Gores G J. Cholangiocarcinoma 2020: the next horizon in mechanisms and management. Nat Rev Gastroenterol Hepatol. 2020; 17(9):557-88. Epub 2020/07/02. doi: 10.1038/s41575-020-0310-z. PubMed PMID: 32606456; PMCID: PMC7447603.
15. Navaneethan U, Njei B, Lourdusamy V, Konjeti R, Vargo J J, Parsi M A. Comparative effectiveness of biliary brush cytology and intraductal biopsy for detection of malignant biliary strictures: a systematic review and meta-analysis. Gastrointest Endosc. 2015; 81(1):168-76. Epub 2014/12/03. doi: 10.1016/j.gie.2014.09.017. PubMed PMID: 25440678; PMCID: PMC4824293.
16. Tabibian J H, Lindor K D. Challenges of Cholangiocarcinoma Detection in Patients with Primary Sclerosing Cholangitis. J Anal Oncol. 2012; 1(1):50-5. Epub 2012/01/01. doi: 10.6000/1927-7229.2012.01.01.7. PubMed PMID: 31897266; PMCID: PMC6939639.
17. Zhang F, Zhang Y, Zhao W, Deng K, Wang Z, Yang C, Ma L, Openkova M S, Hou Y, Li K. Metabolomics for biomarker discovery in the diagnosis, prognosis, survival and recurrence of colorectal cancer: a systematic review. Oncotarget. 2017; 8(21):35460-72. Epub 2017/04/09. doi: 10.18632/oncotarget.16727. PubMed PMID: 28389626; PMCID: PMC5471069.
18. Long N P, Yoon S J, Anh N H, Nghi T D, Lim D K, Hong Y J, Hong S S, Kwon S W. A systematic review on metabolomics-based diagnostic biomarker discovery and validation in pancreatic cancer. Metabolomics. 2018; 14(8):109. Epub 2019/03/05. doi: 10.1007/s11306-018-1404-2. PubMed PMID: 30830397.
19. Xia J, Broadhurst D I, Wilson M, Wishart D S. Translational biomarker discovery in clinical metabolomics: an introductory tutorial. Metabolomics. 2013; 9(2):280-99. Epub 2013/04/02. doi: 10.1007/s11306-012-0482-9. PubMed PMID: 23543913; PMCID: PMC3608878.
20. Swidsinski A, Ludwig W, Pahlig H, Priem F. Molecular genetic evidence of bacterial colonization of cholesterol gallstones. Gastroenterology. 1995; 108(3):860-4. Epub 1995/03/01. doi: 10.1016/0016-5085(95)90461-1. PubMed PMID: 7875489.
21. Gregg J A, De Girolami P, Carr-Locke D L. Effects of sphincteroplasty and endoscopic sphincterotomy on the bacteriologic characteristics of the common bile duct. Am J Surg. 1985; 149(5):668-71. Epub 1985/05/01. doi: 10.1016/s0002-9610(85)80152-5. PubMed PMID: 3993851.
22. Tietz-Bogert P S, Kim M, Cheung A, Tabibian J H, Heimbach J K, Rosen C B, Nandakumar M, Lazaridis K N, LaRusso N F, Sung J, O'Hara S P. Metabolomic Profiling of Portal Blood and Bile Reveals Metabolic Signatures of Primary Sclerosing Cholangitis. Int J Mol Sci. 2018; 19(10). Epub 2018/10/20. doi: 10.3390/ijms19103188. PubMed PMID: 30332763; PMCID: PMC6214107.
23. Martinez-Reyes I, Cardona L R, Kong H, Vasan K, McElroy G S, Werner M, Kihshen H, Reczek C R, Weinberg S E, Gao P, Steinert E M, Piseaux R, Budinger G R S, Chandel N S. Mitochondrial ubiquinol oxidation is necessary for tumour growth. Nature. 2020; 585(7824):288-92. Epub 2020/07/10. doi: 10.1038/s41586-020-2475-6. PubMed PMID: 32641834; PMCID: PMC7486261.
24. Gail M H, Wan Y, Shi J. Power of Microbiome Beta-Diversity Analyses Based on Standard Reference Samples. Am J Epidemiol. 2021; 190(3):439-47. Epub 2020/09/26. doi: 10.1093/aje/kwaa204. PubMed PMID: 32976571; PMCID: PMC7936037.
25. Younge N, McCann J R, Ballard J, Plunkett C, Akhtar S, Araujo-Perez F, Murtha A, Brandon D, Seed P C. Fetal exposure to the maternal microbiota in humans and mice. JCI Insight. 2019; 4(19). Epub 2019/09/04. doi: 10.1172/jci.insight.127806. PubMed PMID: 31479427; PMCID: PMC6795398.
26. Bernini P, Bertini I, Luchinat C, Nepi S, Saccenti E, Schafer H, Schutz B, Spraul M, Tenori L. Individual human phenotypes in metabolic space and time. J Proteome Res. 2009; 8(9):4264-71. Epub 2009/06/17. doi: 10.1021/pr900344m. PubMed PMID: 19527021.
27. Xia J Y, Holland W L, Kusminski C M, Sun K, Sharma A X, Pearson M J, Sifuentes A J, McDonald J G, Gordillo R, Scherer P E. Targeted Induction of Ceramide Degradation Leads to Improved Systemic Metabolism and Reduced Hepatic Steatosis. Cell Metab. 2015; 22(2):266-78. Epub 2015/07/21. doi: 10.1016/j.cmet.2015.06.007. PubMed PMID: 26190650; PMCID: PMC4527941.

1. A method of assessing a pancreaticobiliary condition in a subject comprising assessing the level of one or more biomarkers in a bile sample from the subject.

2. A method of treating a subject suspected of suffering from an indeterminant biliary stricture, comprising:
(a) assessing the levels of one or more biomarkers in a bile sample from the subject;
(b) (i) if the biomarker level(s) indicate a malignancy, administering ERCP with cholangioscopy, endoscopic ultrasound guided biopsy, interventional radiology biopsy, PET CT to identify metastasis, or seeking further review of the subject, and (ii) if the biomarker level(s) indicate a benign condition, conducting additional imaging.

3. The method of claim 1 or 2, wherein the subject suffers exhibits one or more symptoms of the pancreaticobiliary condition.

4. The method of claim 3, wherein the symptoms of the pancreaticobiliary condition are selected from abdominal pain, loss of appetite, weight loss, jaundice, discolored urine or stool, itchy skin, diabetes, and blood clots.

5. The method of claim 1 or 2, wherein the subject has received test results indicating a possible pancreaticobiliary condition.

6. The method of claim 5, wherein the test results are selected from imaging and a blood test.

7. The method of claim 1 or 2, wherein assessing a pancreaticobiliary condition in a subject comprises determining the likelihood that a subject suffers from a pancreaticobiliary cancer versus a benign or non-cancerous pancreaticobiliary condition.

8. The method of claim 7, wherein a pancreaticobiliary cancer is selected from a cancer of the pancreas gland, pancreas duct, pancreatic ampulla, bile ducts, gallbladder, liver parenchyma, and duodenum.

9. The method of claim 7, wherein a benign or non-cancerous pancreaticobiliary condition is selected from a benign stricture, acute pancreatitis, chronic pancreatitis, pancreatic obstruction, choledocholithiasis, Mirizzi syndrome, primary sclerosing cholangitis, liver transplant related anastomotic stricture, biliary surgery related stricture, biliary anatomic variations, autoimmune pancreatitis, and cholangitis.

10. The method of claim 1 or 2, wherein the one or more biomarkers are selected from microbiomic, metabolomic, and/or glycomic biomarkers.

11. The method of claim 10, wherein one or more microbiomic biomarkers, selected from the level of bacteria belonging to one or more genera selected from Peptostreptococcus, Neisseria, Absconditabacteria (SR1), Leptotrichia, Gemella, Lachnospiraceae, Lachnoanaerobaculum, Moigibacterium, Megasphaera, Comamonadaceae, Enhydrobacter, Escherichia-Shigella, Enterococcus, Streptococcus, Actinomyces, and Veillonella.

12. The method of claim 11, wherein an increased level of one or more of Peptostreptococcus, Neisseria, Absconditabacteria (SR1), Leptotrichia, Gemella, Lachnospiraceae, Lachnoanaerobaculum, and/or Moigibacterium is associated with increased risk of one or more pancreaticobiliary cancers.

13. The method of claim 11, wherein a decreased level of one or more of Megasphaera, Comamonadaceae, Enhydrobacter, Escherichia-Shigella, Enterococcus, Streptococcus, Actinomyces, Veillonella is associated with increased risk of one or more pancreaticobiliary cancers.

14. The method of claim 11, wherein upregulation of one or more of 3-/7-methylguanine, sucrose, 5-methylcytosine, tryptamine, N-methyltryptamine, 1-phenylethylamine, pyruvic acid, deoxyadenosine, deoxycytidine triphosphate (dCTP), picolinic acid, guanidinoacetate, NADPH, 5′methylthioadenosine, deoxyuridine, D-sedoheptulose-7-phoisphate, aspartic acid, lactose/maltose, uric acid, phenylalanine, glutamate, uridine monophosphate (UMP), uracil, and xanthine is associated with increased risk of one or more pancreaticobiliary cancers.

15. The method of claim 10, wherein the level of one or more microbiomic biomarkers is assessed by nucleic acid sequencing.

16. The method of claim 10, wherein the level of one or more metabolomic biomarkers is assessed by one or more biophysical techniques.

17. The method of claim 16, wherein the level of one or more metabolomic biomarkers is assessed by mass spectrometric analysis of the bile sample from the subject.

18. The method of claim 10, wherein the one or more metabolomic biomarkers and selected from the level of urea, norepinephrine, pyroglutamic acid, N-acetyl-DL-alanine, levulinic acid, uracil, hydroxyphenyllactic, 4-trimethylammoniobutanoate, ornithine, dihydroorotate, GalNAC/Glc/NAC/ManNAC, taurine, fructose mannose, AICA ribonucleotide, carbamoyl phosphate, 2-keto-isovaleric acid, N-acetyle-DL-serine, galactosamine/glucosamine, aconitic acid, mesaconic acid, itaconic acid, b-alanine, cystosine, lysine, citrate, methylguanine, phenethelamine, 3,4-Dihydroxyphenylacetic acid, D-galactonic acid, carnitine, quinolinic acid, orotic acid, lactic acid, dehydroascorbic acid, xanthine, hypoxanthine, guanine, glycine, 3-methoxytyramine, DL-DOPA, pantothenic acid, succinic acid, 3-aminoisobutanoate, y-glutamylcysteine, alpha-ketoglutarate, 3-phosphonatooxypyruvate, glyceric acid, alanine, creatine, 3-/7-methylguanine, sucrose, 5-methylcytosine, tryptamine, N-methyltryptamine, 1-phenylethylamine, pyruvic acid, deoxyadenosine, deoxycytidine triphosphate (dCTP), picolinic acid, guanidinoacetate, NADPH, 5′methylthioadenosine, deoxyuridine, D-sedoheptulose-7-phoisphate, aspartic acid, lactose/maltose, uric acid, phenylalanine, glutamate, uridine monophosphate (UMP), uracil, and xanthine.

19. The method of claim 18, wherein an increased level of one or more of urea, norepinephrine, pyroglutamic acid, N-acetyl-DL-alanine, levulinic acid, uracil, hydroxyphenyllactic, 4-trimethylammoniobutanoate, ornithine, dihydroorotate, GalNAC/Glc/NAC/ManNAC, taurine, fructose mannose, AICA ribonucleotide, carbamoyl phosphate, 2-keto-isovaleric acid, N-acetyle-DL-serine, galactosamine/glucosamine, aconitic acid, mesaconic acid, itaconic acid, b-alanine, cystosine, lysine, citrate, methylguanine, phenethelamine, 3,4-Dihydroxyphenylacetic acid, D-galactonic acid, carnitine, quinolinic acid, orotic acid, lactic acid, dehydroascorbic acid, xanthine, hypoxanthine, and/or guanine is associated with increased risk of one or more pancreaticobiliary cancers.

20. The method of claim 18, wherein a decreased level of one or more of glycine, 3-methoxytyramine, DL-DOPA, pantothenic acid, succinic acid, 3-aminoisobutanoate, y-glutamylcysteine, alpha-ketoglutarate, 3-phosphonatooxypyruvate, glyceric acid, alanine, and creatine is associated with increased risk of one or more pancreaticobiliary cancers.

21. The method of claim 18, wherein upregulation of one or more of 3-/7-methylguanine, sucrose, 5-methylcytosine, tryptamine, N-methyltryptamine, 1-phenylethylamine, pyruvic acid, deoxyadenosine, deoxycytidine triphosphate (dCTP), picolinic acid, guanidinoacetate, NADPH, 5′methylthioadenosine, deoxyuridine, D-sedoheptulose-7-phoisphate, aspartic acid, lactose/maltose, uric acid, phenylalanine, glutamate, uridine monophosphate (UMP), uracil, and xanthine is associated with increased risk of one or more pancreaticobiliary cancers.

22. The method of claim 10, wherein the level of one or more glycomic biomarkers is assessed by one or more biophysical techniques.

23. The method of claim 22, wherein the level of one or more glycomic biomarkers is assessed by mass spectrometric analysis of the bile sample from the subject.

24. The method of claim 23, wherein the one or more glycomic biomarkers and selected from HexNAc₄Hex₄Fuc₃, HexNAc4Hex5NeuAc1, HexNAc4Hex4Fuc4, HexNAc5Hex4Fuc3, HexNAc4Hex5NeuAc2, HexNAc5Hex4Fuc4, HexNAc3Hex3Fuc2, HexNAc4Hex2Fuc2, HexNAc4Hex3Fuc1, HexNAc4Hex4, HexNAc5Hex3, HexNAc3Hex2NeuAc1Fuc2, HexNAc3Hex3Fuc3, HexNAc3Hex3NeuAc1Fuc1, HexNAc2Hex4Fuc3, HexNAc4Hex3Fuc2, HexNAc4Hex4Fuc1, HexNAc5Hex3Fuc1, HexNAc3Hex3Fuc4, HexNAc3Hex3NeuAc1Fuc2, HexNAc4Hex3Fuc3, HexNAc4Hex4Fuc2, HexNAc5Hex3Fuc2, HexNAc5Hex4Fuc1, HexNAc4Hex3Fuc4, HexNAc4Hex3NeuAc1Fuc2, HexNAc2Hex2NeuAc1, HexNAc2Hex3Fuc1, HexNAc3Hex2Fuc1, HexNAc3Hex3Fuc1, HexNAc4Hex2, HexNAc2Hex2Fuc3, HexNAc2Hex2NeuAc1Fuc1, HexNAc2Hex3Fuc2, HexNAc3Hex2Fuc2, HexNAc3Hex2NeuAc1, HexNAc3Hex3Fuc1, HexNAc1Hex3NeuAc1Fuc2, HexNAc1Hex3NeuAc1, HexNAc4Hex3, HexNAc2Hex2NeuAc1Fuc2, HexNAc2Hex2NeuAc2, HexNAc2Hex3NeuAc1Fuc1, HexNAc2Hex4Fuc2, HexNAc3Hex2Fuc3, HexNAc3Hex2NeuAc1Fuc1, HexNAc1Hex1, HexNAc2, HexNAc1NeuAc1, HexNAc1Hex1Fuc1, HexNAc2Hex1, HexNAc1Hex1NeuAc1, HexNAc2NeuAc1, HexNAc2Hex1Fuc1, HexNAc2Hex2, HexNAc3Hex1, HexNAc1Hex1NeuAc1Fuc1, HexNAc1Hex2Fuc2, HexNAc2Hex1NeuAc1, HexNAc2Hex2Fuc1, HexNAc3Hex1Fuc1, HexNAc3Hex2, HexNAc1Hex1NeuAc2, HexNAc1Hex3NeuAc1, HexNAc2Hex1NeuAc1Fuc1, HexNAc2Hex2Fuc2, (Hex)3 (HexNAc)3 (Deoxyhexose)1 (NeuAc)3+(Man)3(GlcNAc)2, (Hex)3 (HexNAc)3 (Deoxyhexose)2 (NeuAc)3+(Man)3(GlcNAc)2, (Hex)4 (HexNAc)4 (NeuAc)3+(Man)3(GlcNAc)2, (Hex)4 (HexNAc)4 (Deoxyhexose)1 (NeuAc)3+(Man)3(GlcNAc)2, (Hex)4 (HexNAc)4 (NeuAc)4+(Man)3(GlcNAc)2, (Hex)4 (HexNAc)4 (Deoxyhexose)1 (NeuAc)4+(Man)3(GlcNAc)2, (Hex)2 (HexNAc)2 (NeuAc)1 (NeuGc)1+(Man)3(GlcNAc)2, (Hex)2 (HexNAc)3 (Deoxyhexose)1 (NeuAc)1+, (Man)3(GlcNAc)2, (Hex)3 (HexNAc)3 (NeuAc)1+(Man)3(GlcNAc)2, (Hex)2 (HexNAc)4 (Deoxyhexose)2+(Man)3(GlcNAc)2, (Hex)2 (HexNAc)2 (Deoxyhexose)3 (NeuAc)1+(Man)3(GlcNAc)2, (Hex)2 (HexNAc)2 (Deoxyhexose)1 (NeuAc)2+(Man)3(GlcNAc)2, (Hex)3 (HexNAc)2 (NeuAc)2+(Man)3(GlcNAc)2 (Hex)2 (HexNAc)3 (Deoxyhexose)4+(Man)3(GlcNAc)2, (Hex)2 (HexNAc)3 (Deoxyhexose)2 (NeuAc)1+(Man)3(GlcNAc)2, (Hex)3 (HexNAc)3 (Deoxyhexose)1 (NeuAc)1+(Man)3(GlcNAc)2, (Hex)2 (HexNAc)4 (Deoxyhexose)3+(Man)3(GlcNAc)2, (Hex)2 (HexNAc)3 (Deoxyhexose)3 (NeuAc)1+(Man)3(GlcNAc)2, (Hex)2 (HexNAc)3 (Deoxyhexose)1 (NeuAc)2+(Man)3(GlcNAc)2, (Hex)3 (HexNAc)3 (NeuAc)2+(Man)3(GlcNAc)2, (Hex)3 (HexNAc)4 (Deoxyhexose)3+(Man)3(GlcNAc)2, (Hex)3 (HexNAc)3 (Deoxyhexose)1 (NeuAc)2+(Man)3(GlcNAc)2, (Hex)3 (HexNAc)4 (Deoxyhexose)4+(Man)3(GlcNAc)2, (Hex)3 (HexNAc)3 (NeuAc)3+(Man)3(GlcNAc)2, (Hex)3 (HexNAc)4 (Deoxyhexose)3 (NeuAc)1+(Man)3(GlcNAc)2 (Hex)4 (HexNAc)4 (NeuAc)2+(Man)3(GlcNAc)2, (Hex)6+(Man)3(GlcNAc)2, (Hex)1 (HexNAc)2 (Deoxyhexose)1 (NeuAc)1+(Man)3(GlcNAc)2, (Hex)2 (HexNAc)2 (NeuAc)1+(Man)3(GlcNAc)2, (Hex)1 (HexNAc)3 (Deoxyhexose)2+(Man)3(GlcNAc)2, (Hex)1 (HexNAc)3 (NeuAc)1+(Man)3(GlcNAc)2, (Hex)2 (HexNAc)3 (Deoxyhexose)1+(Man)3(GlcNAc)2, (Hex)2 (HexNAc)1 (NeuAc)2+(Man)3(GlcNAc)2, (Hex)2 (HexNAc)2 (Deoxyhexose)1 (NeuAc)1+(Man)3(GlcNAc)2, (Hex)1 (HexNAc)3 (Deoxyhexose)3+(Man)3(GlcNAc)2, (Hex)1 (HexNAc)3 (Deoxyhexose)1 (NeuAc)1+(Man)3(GlcNAc)2, (Hex)2 (HexNAc)3 (Deoxyhexose)2+(Man)3(GlcNAc)2, (Hex)2 (HexNAc)3 (NeuAc)1+(Man)3(GlcNAc)2, (Hex)5 (HexNAc)2+(Man)3(GlcNAc)2, (Hex)2 (HexNAc)1 (Deoxyhexose)1 (NeuAc)2+(Man)3(GlcNAc)2, (Hex)1 (HexNAc)5 (Deoxyhexose)5+(Man)3(GlcNAc)2, (Hex)1 (HexNAc)2 (Deoxyhexose)1 (NeuAc)2+(Man)3(GlcNAc)2, (Hex)1 (HexNAc)5 (Deoxyhexose)1+(Man)3(GlcNAc)2, (Hex)2 (HexNAc)2 (NeuAc)2+(Man)3(GlcNAc)2, (Hex)2 (HexNAc)5+(Man)3(GlcNAc)2, (Hex)3 (HexNAc)2 (Deoxyhexose)1 (NeuAc)1+(Man)3(GlcNAc)2, (Hex)2 (HexNAc)2, (Man)3(GlcNAc)2, (Hex)2+(Man)3(GlcNAc)2, (HexNAc)2+(Man)3(GlcNAc)2, (Hex)3+(Man)3(GlcNAc)2, (HexNAc)2 (Deoxyhexose)1+(Man)3(GlcNAc)2, (HexNAc)3+(Man)3(GlcNAc)2, (Hex)1 (HexNAc)1 (NeuAc)1+(Man)3(GlcNAc)2, (Hex)4+(Man)3(GlcNAc)2, (Hex)1 (HexNAc)2 (Deoxyhexose)1+(Man)3(GlcNAc)2, (Hex)2 (HexNAc)2+(Man)3(GlcNAc)2, (HexNAc)3 (Deoxyhexose)1+(Man)3(GlcNAc)2, (Hex)1 (HexNAc)3+(Man)3(GlcNAc)2, (Hex)2 (HexNAc)1 (NeuAc)1+(Man)3(GlcNAc)2, (Hex)5+(Man)3(GlcNAc)2, (Hex)1 (HexNAc)2 (Deoxyhexose)2+(Man)3(GlcNAc)2, (Hex)1 (HexNAc)2 (NeuAc)1+(Man)3(GlcNAc)2, (Hex)2 (HexNAc)2 (Deoxyhexose)1+(Man)3(GlcNAc)2, (Hex)1 (HexNAc)3 (Deoxyhexose)1+(Man)3(GlcNAc)2, and (Hex)3 (HexNAc)1 (NeuAc)1+(Man)3(GlcNAc)2.

25. The method of claim 24, wherein an increased level of one or more of HexNAc4Hex4Fuc4, HexNAc5Hex3Fuc2, and/or HexNAc3Hex2Fuc3 is associated with increased risk of one or more pancreaticobiliary cancers.

26. The method of claim 1 or 2, wherein the level of one or more biomarkers in the bile sample are compared to a threshold level.

27. The method of claim 1 or 2, wherein the level of one or more biomarkers in the bile sample are compared to previous levels for the subject.

28. The method of claim 1 or 2, further comprising obtaining the bile sample from the subject.

29. The method of claim 28, wherein the bile sample is obtained during endoscopic retrograde cholangiopancreatography (ERCP) duodenal aspirate, percutaneous biliary drain, percutaneous gallbladder drain, or a T-tube.

30. The method of claim 28, wherein the bile sample is a fluid biopsy sample.

31. The method of claim 30, wherein the bile sample is obtained during a procedure to obtain a solid biopsy sample of tissue and/or cells from the subject.

32. The method of claim 28, wherein the bile sample is obtained without a solid biopsy sample.

33. The method of claim 28, wherein the bile sample is aspirated using a sphincterotome after deep bile cannulation.

34. The method of claim 24, wherein the bile sample is 0.1 to 5.0 ml in volume.

35. The method of claim 1 or 2, wherein assessing the pancreaticobiliary condition in the subject further comprises imaging, endoscopic ultrasound, fluorescence in-situ hybridization, cytology, and/or histology.

36. The method of claim 1, further comprising treating a subject for the pancreaticobiliary condition.

37. The method of claim 36, wherein assessing the pancreaticobiliary condition indicates that the subject suffers from a pancreaticobiliary and the subject is treated for the pancreaticobiliary cancer.

38. The method of claim 37, wherein treating the subject for the pancreaticobiliary cancer comprises surgery, radiation treatment, and/or administering a chemotherapeutic or immunotherapeutic.

39. The method of claim 36, wherein assessing the pancreaticobiliary condition indicates that the subject does not suffer from a pancreaticobiliary and the subject is treated for a benign and/or non-cancerous pancreaticobiliary condition.

40. The method of claim 2, wherein additional imaging is selected from magnetic resonance imaging (MRI) and magnetic resonance cholangiopancreatography (MRCP).

41. The method of claim 2, wherein seeking further review of the subject comprises seeking further review from a transplant surgeon, an oncology specialist, and/or a multi-disciplinary tumor board.

42. A method comprising assessing the level of three or more biomarkers in a bile sample from the subject, the biomarkers selected from:
(a) one or more metabolomic biomarkers selected from the level of urea, norepinephrine, pyroglutamic acid, N-acetyl-DL-alanine, levulinic acid, uracil, hydroxyphenyllactic, 4-trimethylammoniobutanoate, ornithine, dihydroorotate, GalNAC/Glc/NAC/ManNAC, taurine, fructose mannose, AICA ribonucleotide, carbamoyl phosphate, 2-keto-isovaleric acid, N-acetyle-DL-serine, galactosamine/glucosamine, aconitic acid, mesaconic acid, itaconic acid, b-alanine, cystosine, lysine, citrate, methylguanine, phenethelamine, 3,4-Dihydroxyphenylacetic acid, D-galactonic acid, carnitine, quinolinic acid, orotic acid, lactic acid, dehydroascorbic acid, xanthine, hypoxanthine, guanine, glycine, 3-methoxytyramine, DL-DOPA, pantothenic acid, succinic acid, 3-aminoisobutanoate, y-glutamylcysteine, alpha-ketoglutarate, 3-phosphonatooxypyruvate, glyceric acid, alanine, creatine, 3-/7-methylguanine, sucrose, 5-methylcytosine, tryptamine, N-methyltryptamine, 1-phenylethylamine, pyruvic acid, deoxyadenosine, deoxycytidine triphosphate (dCTP), picolinic acid, guanidinoacetate, NADPH, 5′methylthioadenosine, deoxyuridine, D-sedoheptulose-7-phoisphate, aspartic acid, lactose/maltose, uric acid, phenylalanine, glutamate, uridine monophosphate (UMP), uracil, and xanthine;
(b) one or more microbiomic biomarkers selected from the level of bacteria belonging to one or more genera selected from Peptostreptococcus, Neisseria, Absconditabacteria (SR1), Leptotrichia, Gemella, Lachnospiraceae, Lachnoanaerobaculum, Moigibacterium, Megasphaera, Comamonadaceae, Enhydrobacter, Escherichia-Shigella, Enterococcus, Streptococcus, Actinomyces, and Veillonella; and/or
(c) one or more glycomic biomarkers and selected from HexNAc₄Hex₄Fuc₃, HexNAc4Hex5NeuAc1, HexNAc4Hex4Fuc4, HexNAc5Hex4Fuc3, HexNAc4Hex5NeuAc2, HexNAc5Hex4Fuc4, HexNAc3Hex3Fuc2, HexNAc4Hex2Fuc2, HexNAc4Hex3Fuc1, HexNAc4Hex4, HexNAc5Hex3, HexNAc3Hex2NeuAc1Fuc2, HexNAc3Hex3Fuc3, HexNAc3Hex3NeuAc1Fuc1, HexNAc2Hex4Fuc3, HexNAc4Hex3Fuc2, HexNAc4Hex4Fuc1, HexNAc5Hex3Fuc1, HexNAc3Hex3Fuc4, HexNAc3Hex3NeuAc1Fuc2, HexNAc4Hex3Fuc3, HexNAc4Hex4Fuc2, HexNAc5Hex3Fuc2, HexNAc5Hex4Fuc1, HexNAc4Hex3Fuc4, HexNAc4Hex3NeuAc1Fuc2, HexNAc2Hex2NeuAc1, HexNAc2Hex3Fuc1, HexNAc3Hex2Fuc1, HexNAc3Hex3Fuc1, HexNAc4Hex2, HexNAc2Hex2Fuc3, HexNAc2Hex2NeuAc1Fuc1, HexNAc2Hex3Fuc2, HexNAc3Hex2Fuc2, HexNAc3Hex2NeuAc1, HexNAc3Hex3Fuc1, HexNAc1Hex3NeuAc1Fuc2, HexNAc1Hex3NeuAc1, HexNAc4Hex3, HexNAc2Hex2NeuAc1Fuc2, HexNAc2Hex2NeuAc2, HexNAc2Hex3NeuAc1Fuc1, HexNAc2Hex4Fuc2, HexNAc3Hex2Fuc3, HexNAc3Hex2NeuAc1Fuc1, HexNAc1Hex1, HexNAc2, HexNAc1NeuAc1, HexNAc1Hex1Fuc1, HexNAc2Hex1, HexNAc1Hex1NeuAc1, HexNAc2NeuAc1, HexNAc2Hex1Fuc1, HexNAc2Hex2, HexNAc3Hex1, HexNAc1Hex1NeuAc1Fuc1, HexNAc1Hex2Fuc2, HexNAc2Hex1NeuAc1, HexNAc2Hex2Fuc1, HexNAc3Hex1Fuc1, HexNAc3Hex2, HexNAc1Hex1NeuAc2, HexNAc1Hex3NeuAc1, HexNAc2Hex1NeuAc1Fuc1, HexNAc2Hex2Fuc2, (Hex)3 (HexNAc)3 (Deoxyhexose)1 (NeuAc)3+(Man)3(GlcNAc)2, (Hex)3 (HexNAc)3 (Deoxyhexose)2 (NeuAc)3+(Man)3(GlcNAc)2, (Hex)4 (HexNAc)4 (NeuAc)3+(Man)3(GlcNAc)2, (Hex)4 (HexNAc)4 (Deoxyhexose)1 (NeuAc)3+(Man)3(GlcNAc)2, (Hex)4 (HexNAc)4 (NeuAc)4+(Man)3(GlcNAc)2, (Hex)4 (HexNAc)4 (Deoxyhexose)1 (NeuAc)4+(Man)3(GlcNAc)2, (Hex)2 (HexNAc)2 (NeuAc)1 (NeuGc)1+(Man)3(GlcNAc)2, (Hex)2 (HexNAc)3 (Deoxyhexose)1 (NeuAc)1+, (Man)3(GlcNAc)2, (Hex)3 (HexNAc)3 (NeuAc)1+(Man)3(GlcNAc)2, (Hex)2 (HexNAc)4 (Deoxyhexose)2+(Man)3(GlcNAc)2, (Hex)2 (HexNAc)2 (Deoxyhexose)3 (NeuAc)1+(Man)3(GlcNAc)2, (Hex)2 (HexNAc)2 (Deoxyhexose)1 (NeuAc)2+(Man)3(GlcNAc)2, (Hex)3 (HexNAc)2 (NeuAc)2+(Man)3(GlcNAc)2 (Hex)2 (HexNAc)3 (Deoxyhexose)4+(Man)3(GlcNAc)2, (Hex)2 (HexNAc)3 (Deoxyhexose)2 (NeuAc)1+(Man)3(GlcNAc)2, (Hex)3 (HexNAc)3 (Deoxyhexose)1 (NeuAc)1+(Man)3(GlcNAc)2, (Hex)2 (HexNAc)4 (Deoxyhexose)3+(Man)3(GlcNAc)2, (Hex)2 (HexNAc)3 (Deoxyhexose)3 (NeuAc)1+(Man)3(GlcNAc)2, (Hex)2 (HexNAc)3 (Deoxyhexose)1 (NeuAc)2+(Man)3(GlcNAc)2, (Hex)3 (HexNAc)3 (NeuAc)2+(Man)3(GlcNAc)2, (Hex)3 (HexNAc)4 (Deoxyhexose)3+(Man)3(GlcNAc)2, (Hex)3 (HexNAc)3 (Deoxyhexose)1 (NeuAc)2+(Man)3(GlcNAc)2, (Hex)3 (HexNAc)4 (Deoxyhexose)4+(Man)3(GlcNAc)2, (Hex)3 (HexNAc)3 (NeuAc)3+(Man)3(GlcNAc)2, (Hex)3 (HexNAc)4 (Deoxyhexose)3 (NeuAc)1+(Man)3(GlcNAc)2 (Hex)4 (HexNAc)4 (NeuAc)2+(Man)3(GlcNAc)2, (Hex)6+(Man)3(GlcNAc)2, (Hex)1 (HexNAc)2 (Deoxyhexose)1 (NeuAc)1+(Man)3(GlcNAc)2, (Hex)2 (HexNAc)2 (NeuAc)1+(Man)3(GlcNAc)2, (Hex)1 (HexNAc)3 (Deoxyhexose)2+(Man)3(GlcNAc)2, (Hex)1 (HexNAc)3 (NeuAc)1+(Man)3(GlcNAc)2, (Hex)2 (HexNAc)3 (Deoxyhexose)1+(Man)3(GlcNAc)2, (Hex)2 (HexNAc)1 (NeuAc)2+(Man)3(GlcNAc)2, (Hex)2 (HexNAc)2 (Deoxyhexose)1 (NeuAc)1+(Man)3(GlcNAc)2, (Hex)1 (HexNAc)3 (Deoxyhexose)3+(Man)3(GlcNAc)2, (Hex)1 (HexNAc)3 (Deoxyhexose)1 (NeuAc)1+(Man)3(GlcNAc)2, (Hex)2 (HexNAc)3 (Deoxyhexose)2+(Man)3(GlcNAc)2, (Hex)2 (HexNAc)3 (NeuAc)1+(Man)3(GlcNAc)2, (Hex)5 (HexNAc)2+(Man)3(GlcNAc)2, (Hex)2 (HexNAc)1 (Deoxyhexose)1 (NeuAc)2+(Man)3(GlcNAc)2, (Hex)1 (HexNAc)5 (Deoxyhexose)5+(Man)3(GlcNAc)2, (Hex)1 (HexNAc)2 (Deoxyhexose)1 (NeuAc)2+(Man)3(GlcNAc)2, (Hex)1 (HexNAc)5 (Deoxyhexose)1+(Man)3(GlcNAc)2, (Hex)2 (HexNAc)2 (NeuAc)2+(Man)3(GlcNAc)2, (Hex)2 (HexNAc)5+(Man)3(GlcNAc)2, (Hex)3 (HexNAc)2 (Deoxyhexose)1 (NeuAc)1+(Man)3(GlcNAc)2, (Hex)2 (HexNAc)2, (Man)3(GlcNAc)2, (Hex)2+(Man)3(GlcNAc)2, (HexNAc)2+(Man)3(GlcNAc)2, (Hex)3+(Man)3(GlcNAc)2, (HexNAc)2 (Deoxyhexose)1+(Man)3(GlcNAc)2, (HexNAc)3+(Man)3(GlcNAc)2, (Hex)1 (HexNAc)1 (NeuAc)1+(Man)3(GlcNAc)2, (Hex)4+(Man)3(GlcNAc)2, (Hex)1 (HexNAc)2 (Deoxyhexose)1+(Man)3(GlcNAc)2, (Hex)2 (HexNAc)2+(Man)3(GlcNAc)2, (HexNAc)3 (Deoxyhexose)1+(Man)3(GlcNAc)2, (Hex)1 (HexNAc)3+(Man)3(GlcNAc)2, (Hex)2 (HexNAc)1 (NeuAc)1+(Man)3(GlcNAc)2, (Hex)5+(Man)3(GlcNAc)2, (Hex)1 (HexNAc)2 (Deoxyhexose)2+(Man)3(GlcNAc)2, (Hex)1 (HexNAc)2 (NeuAc)1+(Man)3(GlcNAc)2, (Hex)2 (HexNAc)2 (Deoxyhexose)1+(Man)3(GlcNAc)2, (Hex)1 (HexNAc)3 (Deoxyhexose)1+(Man)3(GlcNAc)2, and (Hex)3 (HexNAc)1 (NeuAc)1+(Man)3(GlcNAc)2.

43. A method comprising assessing the level of three or more of 3-methylguanine, 5-methylcytosine, butyric acid, and urea in a bile sample from the subject.
